(19) United States
(12) Patent Application Publication
(10) Pub. No.: US 2026/0306671 A1
(43) Pub. Date: Oct. 8, 2026

(54) TRANSCRIPTION FACTOR 4 GENE EDITING SYSTEM

(71) Applicant: THE REGENTS OF THE UNIVERSITY OF CALIFORNIA, Oakland, CA (US)

(72) Inventors: Katherine Lindsay LUCOT SMITH, Davis, CA (US); Kyle FINK, Davis, CA (US); Julian HALMAI, Davis, CA (US); Renee Ellen NAPOLIELLO, Davis, CA (US)

(21) Appl. No.: 19/483,254

(22) PCT Filed: May 15, 2024
(86) PCT No.: PCT/US2024/029527

§ 371 Date: Nov. 11, 2025

Related U.S. Application Data
(60) Provisional application No. 63/502,265, filed on May 15, 2023.

(51) Int. Cl. A61K 48/00; A61K 38/17; A61P 25/28; C12N 9/22; C12N 15/11; C12N 15/86

(52) U.S. Cl. CPC A61K 48/005; A61K 38/1709; A61K 48/0075; A61P 25/28; C12N 9/226; C12N 15/111; C12N 15/86; C07K 2319/71; C12N 2310/20; C12N 2750/14143

(57) ABSTRACT

Pitt-Hopkins Syndrome (PTHS) is a disorder characterized by severe intellectual disability and tendency for epilepsy. There is currently no treatment for PTHS. PTHS is caused by de novo mutations in Transcription Factor 4 (TCF4). No familial cases have been reported and all patients, except for two, have a unique mutation; many of which generate haploinsufficiency. Haploinsufficiency occurs in individual with one mutant allele, and the cell thus fails to meet the dosage of functional protein required for cellular function. Disclosed herein are gene editing systems that can enhance in vivo TCF4 expression.

### CROSS-REFERENCE TO RELATED APPLICATIONS

This application claims benefit of U.S. Provisional Application No. 63/502,265, filed May 15, 2024, which is hereby incorporated herein by reference in its entirety.

### BACKGROUND OF THE INVENTION

Pitt-Hopkins Syndrome (PTHS) is a disorder characterized by severe intellectual disability and tendency for epilepsy. There is currently no treatment for PTHS. PTHS is caused by de novo mutations in Transcription Factor 4 (TCF4). No familial cases have been reported and all patients, except for two, have a unique mutation; many of which generate haploinsufficiency. Haploinsufficiency occurs in individual with one mutant allele, and the cell thus fails to meet the dosage of functional protein required for cellular function. Compositions and methods are needed to improve TCF4 expression during both neural development and adult neuronal function.

### SUMMARY OF THE INVENTION

Disclosed herein is a gene editing system that involves a first nucleotide molecule encoding a catalytically inactive Cas9 (dCas9) protein fused to a miniaturized VP64-p65-Rta (miniVPR) transcriptional activator and further encoding an MS2-P65-HSF1 (MPH) transcriptional activation complex. In some embodiments, the gene editing system further involves a second nucleotide molecule encoding at least one small guide RNA (sgRNA) comprising a scaffold region and a spacer region, wherein the scaffold region comprises MS2 stem loops, wherein the spacer region hybridizes to a nucleotide sequence complementary to a target sequence adjacent to a 5′-end of a protospacer adjacent motif (PAM), and wherein the target sequence and the PAM are located within 1 kilobase (kb) of the transcriptional start site (TSS) of the TCF4 gene.

Also disclosed herein is a gene editing system that involves a first nucleotide molecule encoding a first half of a split catalytically inactive Cas9 (dCas9) protein fused to a mini VP64-p65-Rta (miniVPR) transcriptional activator and a first half of an intein, and a second nucleotide molecule encoding a second half of the split dCas9 fused to a second half of an intein and further encoding an MS2-P65-HSF1 (MPH) transcriptional activation complex. In some embodiments, the gene editing system further involves a third nucleotide molecule encoding at least one small guide RNA (sgRNA) comprising a scaffold region and a spacer region, wherein the scaffold region comprises MS2 stem loops, wherein the spacer region hybridizes to a nucleotide sequence complementary to a target sequence adjacent to a 5′-end of a protospacer adjacent motif (PAM), and wherein the target sequence and the PAM are located within 1 kilobase (kb) of the transcriptional start site (TSS) of the TCF4 gene. In this embodiment, the split dCas9 encoded by the first nucleotide is transpliced with the split dCas9 encoded by the third nucleotide to produce a complete dCas9. For example, in some embodiments, the first nucleotide molecule encodes the N-terminal half of the split dCas9 protein fused to the miniVPR transcriptional activator and the N-terminal half of the intein, and the second nucleotide molecule encodes the C-terminal half of the split dCas9 fused to the C-terminal half of the intein and further encodes the MPH transcriptional activation complex. In other embodiments, the first nucleotide molecule encodes the C-terminal half of the split dCas9 protein fused to the miniVPR transcriptional activator and the C-terminal half of the intein, and the second nucleotide molecule encodes the N-terminal half of the split dCas9 fused to the N-terminal half of the intein and further encodes the MPH transcriptional activation complex.

In some embodiments the spacer region of the sgRNA comprises the nucleic acid sequence GCATCCTAGCCTGCCCGACC (SEQ ID NO:1) or GTGCCTCCGAGGGATGCAAC (SEQ ID NO:2), or a combination thereof.

In some embodiments, each of the nucleotide molecules are integrated into one or more viral or plasmid vectors. For example, the viral vector can be selected from the group of a lentiviral vector, an adeno-associated viral (AAV) vector, or an adenoviral vector.

Also disclosed herein is a vector encoding an sgRNA, wherein the sgRNA comprises a scaffold region and a spacer region, wherein the spacer region comprises the nucleic acid sequence GCATCCTAGCCTGCCCGACC (SEQ ID NO:1) or GTGCCTCCGAGGGATGCAAC (SEQ ID NO:2), or a combination thereof. In some embodiments, the vector is a viral vector or a plasmid vector. For example, the viral vector can be a lentiviral vector, an AAV vector, or an adenoviral vector. In some embodiments, the vector further contains a nucleotide molecule encoding a catalytically inactive Cas9 (dCas9) protein fused to at least one transcriptional activator. In some embodiments, the transcriptional activator comprises miniVPR or a biological active fragment thereof. In some embodiments, the transcriptional activator comprises MS2-P65-HSF1 (MPH) or a biological active fragment thereof. Therefore, in some embodiments, the first and second transcriptional activator are miniVPR and MS2-P65-HSF1 (MPH) or MS2-P65-HSF1 (MPH) and miniVPR, respectively.

In some embodiments, the vector further contains a first nucleotide molecule encoding an N-terminal half of a catalytically inactive Cas9 (dCas9) protein fused to a first transcriptional activator and the N-terminal half an intein, and a second nucleotide molecule encoding an C-terminal half of a catalytically inactive Cas9 (dCas9) protein fused to a second transcriptional activator and the C-terminal half an intein.

Also disclosed herein is a host cell containing a gene editing system disclosed herein. In some embodiments, the host cell is a prokaryotic or a eukaryotic cell. In some embodiments, the host cell is a mammalian or a human cell. In some embodiments, the host cell is a cultured cell or a primary cell. In some embodiments, the cell is a neuronal cell.

In some embodiments, the cell further contains a therapeutic molecule.

A pharmaceutical composition comprising one or more of the gene editing systems, vectors, or host cells disclosed herein in a pharmaceutically acceptable carrier or excipient.

Also disclosed herein is a method for increasing TCF4 gene expression in a cell or subject that involves administering to the cell or subject a pharmaceutical composition disclosed herein. For example, in some embodiments the cell or subject is in need of increased TCF4 gene expression. For example, in some embodiments, the subject has been diagnosed with Pitt-Hopkins Syndrome (PTHS). The pharmaceutical composition can be administered to the subject by one or more of: an intravenous route, a subcutaneous route, an intramuscular route, an intradermal route, an intranasal route, an oral route, an intracranial route, an intrathecal route, an ocular route, an otic route, a rectal route, a vaginal route, an optic route, or an intraperitoneal route. In some embodiments, the subject is a human. In some embodiments, the subject is a fetus, infant, or juvenile.

Also disclosed herein is a method for treating or preventing Pitt-Hopkins Syndrome (PTHS) in a subject in need thereof that involves administering to the subject a pharmaceutical composition disclosed herein. In some embodiments, the pharmaceutical composition is administered to the subject by one or more of: an intravenous route, a subcutaneous route, an intramuscular route, an intradermal route, an intranasal route, an oral route, an intracranial route, an intrathecal route, an ocular route, an otic route, a rectal route, a vaginal route, an optic route, or an intraperitoneal route. In some embodiments, the subject is a human. In some embodiments, the subject is a fetus, infant, or juvenile.

Also disclosed is a kit containing a system, vector, or cell disclosed herein and optional instructions for use in using as described herein.

The details of one or more embodiments of the invention are set forth in the accompanying drawings and the description below. Other features, objects, and advantages of the invention will be apparent from the description and drawings, and from the claims.

### BRIEF DESCRIPTION OF FIGURES

FIG. 1 shows haploinsufficiency from de novo mutations in TCF4.

FIG. 2 illustrates how miniVPR and MPH are powerful transcriptional activators when combined in the same system.

FIG. 3 illustrates a split dCas9 using intein sequences.

FIG. 4 illustrates a transfection protocol.

FIG. 5A shows transcript expression fold change between treatment and control (expression of TCF4 normalized to Beta Actin). Two-way ANOVA with Sidak's multiple comparisons test was performed (p=0.05). FIG. 5B shows transcript expression fold change between treatment and control (expression of TCF4 normalized to Beta Actin). An unpaired t test was performed (p=0.05).

FIG. 6A shows transcript expression fold change between treatment and control (expression of Tcf4 normalized to Gapdh) in transfected neuro-2a cells (mouse neuroblasts). Two-way ANOVA with Sidak's multiple comparisons test was performed (p=0.05). FIG. 6B shows transcript expression fold change in transfected N2 as with n=6 for treatment and control.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

### Definitions

It must be noted that, as used in the specification and the appended claims, the singular forms “a,” “an,” and “the” include plural referents unless the context clearly dictates otherwise.

As used herein, the term “about,” when referring to a measurable value such as an amount or concentration and the like, is meant to encompass variations of 20%, 10%, 5%, 1%, 0.5%, or even 0.1% of the specified amount.

As used herein, the terms or “acceptable,” “effective,” or “sufficient” when used to describe the selection of any components, ranges, dose forms, etc. disclosed herein intend that said component, range, dose form, etc. is suitable for the disclosed purpose.

As used herein, the term “adeno-associated virus” or “AAV” refers to a member of the class of viruses associated with this name and belonging to the genus Dependoparvovirus, family Parvoviridae. Multiple serotypes of this virus are known to be suitable for gene delivery; all known serotypes can infect cells from various tissue types. At least 11 or 12, sequentially numbered, are disclosed in the prior art. Non-limiting exemplary serotypes useful in the gene editing systems, host cells, pharmaceutical compositions, vectors, and methods disclosed herein include any of the 11 or 12 serotypes, e.g., AAV2, AAV5, and AAV8, or variant serotypes, e.g. AAV-DJ. The AAV structural particle is composed of 60 protein molecules made up of VP1, VP2 and VP3. Each particle contains approximately 5 VP1 proteins, 5 VP2 proteins and 50 VP3 proteins ordered into an icosahedral structure.

As used herein, the term “administering” a compound or composition to a subject means delivering the compound to the subject. “Administering” includes prophylactic administration of the compound or composition (i.e., before the disease and/or one or more symptoms of the disease are detectable) and/or therapeutic administration of the composition (i.e., after the disease and/or one or more symptoms of the disease are detectable). The methods of the present technology include administering one or more compounds or agents.

If more than one agent is to be administered, the agents may be administered together at substantially the same time, and/or administered at different times in any order.

Also, the agents of the present technology may be administered before, concomitantly with, and/or after administration of another type of composition or therapeutic procedure (e.g., surgery).

As used herein, “ameliorate,” “ameliorating,” and the like, as used herein, refer to inhibiting, relieving, eliminating, or slowing progression of one or more symptoms.

As used herein, “and/or” refers to and encompasses any and all possible combinations of one or more of the associated listed items, as well as the lack of combinations when interpreted in the alternative (“or”).

As used herein, the term “Cas9” refers to a CRISPR-associated, RNA-guided endonuclease such as Streptococcus pyogenes Cas9 (spCas9) and orthologs and biological equivalents thereof. Biological equivalents of Cas9 include but are not limited to C2c1 from Alicyclobacillus acideterrestris and Cpf1 (which performs cutting functions analogous to Cas9) from various bacterial species including Acidaminococcus spp, and Francisella novicida U112. The Cas9 can be a catalytically inactive Cas9 (dCas9) that lack endonuclease activity. The Cas9 can also be a “split-Cas9” in which Cas9 is split into a C-terminal half of a Cas9 fused to and C-terminal half of an intein and an N-terminal half of a Cas9 fused with a C-terminal half of an intein. See, e.g., U.S. Pat. No. 9,074,199 B1; Zetsche et al., Nat Biotechnol. 33 (2): 139-42 (2015); Wright et al., PNAS 112 (10) 2984-89 (2015).

As used herein, the term “cell” or “host cell” may refer to either a prokaryotic or eukaryotic cell, optionally obtained from a subject or a commercially available source.

As used herein, the term “CRISPR” refers to Clustered Regularly Interspaced Short Palindromic Repeats (CRISPR). CRISPR may also refer to a technique or system of sequence-specific genetic manipulation relying on the CRISPR pathway. A CRISPR recombinant expression system can be programmed to cleave a target polynucleotide using a CRISPR endonuclease and a guide RNA. A CRISPR system can be used to cause double stranded or single stranded breaks in a target polynucleotide. A CRISPR system can also be used to recruit proteins or label a target polynucleotide. In some aspects, CRISPR-mediated gene editing utilizes the pathways of nonhomologous end-joining (NHEJ) or homologous recombination to perform the edits. These applications of CRISPR technology are known and widely practiced in the art. See, e.g., U.S. Pat. No. 8,697,359, and Hsu et al., Cell 156 (6): 1262-1278 (2014).

As used herein, the term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific dose may vary depending on one or more of: the particular agent chosen, the dosing regimen to be followed, whether it is administered in combination with other compounds, timing of administration, the route of administration, and the physical delivery system in which it is carried. The compositions can also be administered in combination with one or more additional compounds. Multiple doses may be administered. Additionally or alternatively, multiple therapeutic compositions or compounds may administered. In the methods described herein, the compounds may be administered to a subject having one or more signs or symptoms of a disease or disorder described herein.

As used herein, the term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “variant thereof,” as used with respect to an endonuclease, refers to the referenced endonuclease in its enzymatically functional form expressed in any suitable host organism or expression system and/or including any modifications to enhance the enzymatic activity of the endonuclease.

In some embodiments of the present disclosure, the endonuclease may be a Cas9, Cas13, Cas6, Cpf1, CMS1 protein, or any variant thereof that is derived or expressed from Methanococcus maripaludis C7, Corynebacterium diphtheria, Corynebacterium efficiens YS-314, Corynebacterium glutamicum (ATCC 13032), Corynebacterium glutamicum (ATCC 13032), Corynebacterium glutamicum R, Corynebacterium kroppenstedtii (DSM 44385), Mycobacterium abscessus (ATCC 19977), Nocardia farcinica IFM1 0152, Rhodococcus erythropolis PR4, Rhodococcus jostii RFIA1, Rhodococcus opacus B4 (uid36573), Acidothermus cellulolyticus 11B, Arthrobacter chlorophenolicus A6, Kribbella flavida (DSM 17836, uid43465), Thermomonospora curvata (DSM431 83), Bifidobacterium dentium Bd1, Bifidobacterium longum DJO10A, Slackia heliotrinireducens (DSM 20476), Persephonella marina EX H1, Bacteroides fragilis NCTC 9434, Capnocytophaga ochracea (DSM 7271), Flavobacterium psychrophilum JIP02 86, Akkermansia muciniphila (ATCC BAA 835), Roseiflexus castenholzii (DSM 13941), Roseiflexus RS1, Synechocystis PCC6803, Elusimicrobium minutum Pei1 9 1, uncultured Termite group 1 bacterium phylotype Rs D17, Fibrobacter succinogenes S85, Bacillus cereus (ATCC 10987), Listeria innocua, Lactobacillus casei, Lactobacillus rhamnosus GG, Lactobacillus salivarius UCC1 18, Streptococcus agalactiae-5-A909, Streptococcus agalactiae NEM316, Streptococcus agalactiae 2603, Streptococcus dysgalactiae equisimilis GGS 124, Streptococcus equi zooepidemicus MGCS1 0565, Streptococcus gallolyticus UCN34 (uid46061), Streptococcus gordonii Challis subst CH1, Streptococcus mutans NN2025 (uid46353), Streptococcus mutans, Streptococcus pyogenes M 1 GAS, Streptococcus pyogenes MGAS5005, Streptococcus pyogenes MGAS2096, Streptococcus pyogenes MGAS9429, Streptococcus pyogenes MGAS 10270, Streptococcus pyogenes MGAS61 80, Streptococcus pyogenes MGAS31 5, Streptococcus pyogenes SSI-1, Streptococcus pyogenes MGAS1 0750, Streptococcus pyogenes NZ1 3 1, Streptococcus thermophiles CNRZ1 066, Streptococcus thermophiles LMD-9, Streptococcus thermophiles LMG 1831 1, Clostridium botulinum A3 Loch Maree, Clostridium botulinum B Eklund 17B, Clostridium botulinum Ba4 657, Clostridium botulinum F Langeland, Clostridium cellulolyticum H 10, Finegoldia magna (ATCC 29328), Eubacterium rectale (ATCC 33656), Mycoplasma gallisepticum, Mycoplasma mobile 163K, Mycoplasma penetrans, Mycoplasma synoviae 53, Streptobacillus moniliformis (DSM 121 12), Bradyrhizobium BTAil, Nitrobacter hamburgensis X14, Rhodopseudomonas palustris BisB1 8, Rhodopseudomonas palustris BisB5, Parvibaculum lavamentivorans DS-1, Dinoroseobacter shibae. DFL 12, Gluconacetobacter diazotrophicus Pal 5 FAPERJ, Gluconacetobacter diazotrophicus Pal 5 JGI, Azospirillum B51 0 (uid46085), Rhodospirillum rubrum (ATCC 11170), Diaphorobacter TPSY (uid29975), Verminephrobacter eiseniae EFO1-2, Neisseria meningitides 053442, Neisseria meningitides alpha14, Neisseria meningitides Z2491, Desulfovibrio salexigens DSM 2638, Campylobacter jejuni doylei 269 97, Campylobacter jejuni 8 1116, Campylobacter jejuni, Campylobacter lari RM21 00, Helicobacter hepaticus, Wolinella succinogenes, Tolumonas auensis DSM 9 187, Pseudoalteromonas atlantica T6c, Shewanella pealeana (ATCC 700345), Legionella pneumophila Paris, Actinobacillus succinogenes 130Z, Pasteurella multocida, Francisella tularensis novicida U112, Francisella tularensis holarctica, Francisella tularensis FSC 198, Francisella tularensis, Francisella tularensis WY96-3418, or Treponema denticola (ATCC 35405).

As used herein, the terms “equivalent” or “biological equivalent” are used interchangeably when referring to a particular molecule, biological, or cellular material and intend those having minimal homology while still maintaining desired structure or functionality.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

As used herein, the term “guide polynucleotide” refers to a polynucleotide having a “synthetic sequence” capable of binding the corresponding endonuclease enzyme protein (e.g., Cas9) and a variable target sequence capable of binding the genomic target (e.g., a nucleotide sequence found in an exon of a target gene). In some embodiments of the present disclosure, a guide polynucleotide is a guide ribonucleic acid (gRNA). In some embodiments, the variable target sequence of the guide polynucleotide is any sequence within the target that is unique with respect to the rest of the genome and is immediately adjacent to a Protospacer Adjacent Motif (PAM). The exact sequence of the PAM sequence may vary as different endonucleases require different PAM sequences.

As used herein, “hybridization” or “hybridizes” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme. Examples of stringent hybridization conditions include: incubation temperatures of about 25° C. to about 37° C.; hybridization buffer concentrations of about 6× saline-sodium citrate (“SSC”) to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M sodium chloride (“NaCl”) and 15 mM citrate buffer. It is understood that equivalents of SSC using other buffer systems can be employed.

As used herein, the term “isolated” as used herein refers to molecules or biologicals or cellular materials being substantially free from other materials.

As used herein, the term “lentivirus” refers to a member of the class of viruses associated with this name and belonging to the genus lentivirus, family Retroviridae. While some lentiviruses are known to cause diseases, other lentivirus are known to be suitable for gene delivery. See, e.g., Tomas et al. (2013) Biochemistry, Genetics and Molecular Biology: “Gene Therapy—Tools and Potential Applications,” ISBN 978-953-51-1014-9, DOI: 10.5772/52534.

As used herein, the terms “nucleic acid sequence,” “nucleotide sequence,” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

As used herein, the term “ortholog” is used in reference of another gene or protein and intends a homolog of said gene or protein that evolved from the same ancestral source. Orthologs may or may not retain the same function as the gene or protein to which they are orthologous. Non-limiting examples of Cas9 orthologs include S. aureus Cas9 (“spCas9”), S. thermophiles Cas9, L. pneumophilia Cas9, N. lactamica Cas9, N. meningitides Cas9, B. longum Cas9, A. muciniphila Cas9, and O. laneus Cas9.

As used herein, “prevention,” “prevents,” or “preventing” of a disorder or condition refers to a compound that, in a statistical sample, reduces the occurrence of the disorder, symptom, or condition in the treated sample relative to a control subject, or delays the onset of one or more symptoms of the disorder or condition relative to the control subject.

As used herein, the term “promoter” as used herein refers to any sequence that regulates the expression of a coding sequence, such as a gene. refers to a region of DNA that initiates transcription of a particular gene. The promoter includes the core promoter, which is the minimal portion of the promoter required to properly initiate transcription and can also include regulatory elements such as transcription factor binding sites. The regulatory elements may promote transcription or inhibit transcription. Regulatory elements in the promoter can be binding sites for transcriptional activators or transcriptional repressors. A promoter can be constitutive or inducible. A constitutive promoter refers to one that is always active and/or constantly directs transcription of a gene above a basal level of transcription. An inducible promoter is one which is capable of being induced by a molecule or a factor added to the cell or expressed in the cell. An inducible promoter may still produce a basal level of transcription in the absence of induction, but induction typically leads to significantly more production of the protein. Promoters can also be tissue specific. A tissue specific promoter allows for the production of a protein in a certain population of cells that have the appropriate transcriptional factors to activate the promoter.

Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. Non-limiting exemplary promoters include CDKL5 promoter, SCML2 promoter, COL9A3 promoter, MECP2, CMV promoter and U6 promoter, the phosphoglycerate kinase 1 (PGK) promoter; SSFV, CMV, MNDU3, SV40, Efla, UBC and CAGG. Non-limiting exemplary promoter sequences are provided herein below:

A number of effector elements are disclosed herein for use in these vectors; e.g., a tetracycline response element (e.g., tetO), a tet-regulatable activator, T2A, VP64, RtA, KRAB, and a miRNA sensor circuit. The nature and function of these effector elements are commonly understood in the art and a number of these effector elements are commercially available. Non-limiting exemplary sequences thereof are disclosed herein and further description thereof is provided herein below.

As used herein, the term “protein”, “peptide” and “polypeptide” are used interchangeably and in their broadest sense to refer to a compound of two or more subunits of amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, “protospacer adjacent motif” (PAM) refers to a short nucleotide sequence adjacent to a target sequence (protospacer) that is recognized (targeted) by a sgRNA/Cas endonuclease system described herein. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long. The PAM sequence plays a key role in target recognition by licensing sgRNA base pairing to the protospacer sequence (Szczelkun et al., Proc. Natl. Acad. Sci. U.S.A 111:9798-803 (2014)).

As used herein, the term “recombinant expression system” refers to a genetic construct for the expression of certain genetic material formed by recombination.

As used herein, the term “sgRNA” or “single guide RNA” as used herein refers to the guide RNA sequences used to target specific genes for correction employing the CRISPR technique. Techniques of designing sgRNAs and donor therapeutic polynucleotides for target specificity are well known in the art. For example, Doench et al., Nature Biotechnology 32 (12): 1262-7 (2014), Mohr et al., FEBS J. 283:3232-38 (2016), and Graham et al., Genome Biol. 16:260 (2015). sgRNA comprises or alternatively consists essentially of, or yet further consists of a fusion polynucleotide comprising CRISPR RNA (crRNA; i.e., a scaffold region) and trans-activating CRIPSPR RNA (tracrRNA; i.e., a spacer region); or a polynucleotide comprising crRNA (i.e., a scaffold region) and tracrRNA (i.e., a spacer region). In some aspects, a sgRNA is synthetic (Kelley et al., J of Biotechnology 233:74-83 (2016).

As used herein, the terms “subject,” “individual,” or “patient” can be an individual organism, a vertebrate, a mammal, or a human. “Mammal” includes a human, non-human mammal, non-human primate, murine (e.g., mouse, rat, guinea pig, hamster), ovine, bovine, ruminant, lagomorph, porcine, caprine, equine, canine, feline, avis, etc. In any embodiment herein, the mammal is feline or canine. In any embodiment herein, the mammal is human.

As used herein, “target site” refers to a site of the target sequence including both the target sequence and its complementary sequence, for example, in double stranded nucleotides. The target site described herein may mean a nucleotide sequence hybridizing to a sgRNA spacer region, a complementary nucleotide sequence of the nucleotide sequence hybridizing to a sgRNA spacer region, and/or a nucleotide sequence adjacent to the 5′-end of a PAM. Full complementarity of a sgRNA spacer region with a target site is not necessarily required, provided there is sufficient complementarity to cause hybridization and promote formation of a CRISPR complex. A target sequence or target site may comprise any polynucleotide, such as DNA or RNA polynucleotides. In some embodiments, a target sequence or target site is located in the nucleus or cytoplasm of a cell. In some embodiments, the target sequence or target site may be within an organelle of a eukaryotic cell, for example, mitochondrion or chloroplast.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, the term “treatment” excludes prevention or prophylaxis.

As used herein, “stem cell” defines a cell with the ability to divide for indefinite periods in culture and give rise to specialized cells. At this time and for convenience, stem cells are categorized as somatic (adult) or embryonic. A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell from the embryo that has the potential to become a wide variety of specialized cell types. An embryonic stem cell is one that has been cultured under in vitro conditions that allow proliferation without differentiation for months to years. A clone is a line of cells that is genetically identical to the originating cell; in this case, a stem cell. A population of cells intends a collection of more than one cell that is identical (clonal) or non-identical in phenotype and/or genotype. A substantially homogenous population of cells is a population having at least 70%, or alternatively at least 75%, or alternatively at least 80%, or alternatively at least 85%, or alternatively at least 90%, or alternatively at least 95%, or alternatively at least 98% identical phenotype, as measured by pre-selected markers.

As used herein, “embryonic stem cells” refers to stem cells derived from tissue formed after fertilization but before the end of gestation, including pre-embryonic tissue (such as, for example, a blastocyst), embryonic tissue, or fetal tissue taken any time during gestation, typically but not necessarily before approximately 10-12 weeks gestation. Most frequently, embryonic stem cells are pluripotent cells derived from the early embryo or blastocyst. Embryonic stem cells can be obtained directly from suitable tissue, including, but not limited to human tissue, or from established embryonic cell lines. “Embryonic-like stem cells” refer to cells that share one or more, but not all characteristics, of an embryonic stem cell.

A neural stem cell is a cell that can be isolated from the adult central nervous systems of mammals, including humans. They have been shown to generate neurons, migrate and send out aconal and dendritic projections and integrate into pre-existing neuroal circuits and contribute to normal brain function. Reviews of research in this area are found in Miller (2006) The Promise of Stem Cells for Neural Repair, Brain Res. Vol. 1091 (1): 258-264; Pluchino et al. (2005) Neural Stem Cells and Their Use as Therapeutic Tool in Neurological Disorders, Brain Res. Brain Res. Rev., Vol. 48 (2): 211-219; and Goh, et al. (2003) Adult Neural Stem Cells and Repair of the Adult Central Nervous System, J. Hematother. Stem Cell Res., Vol. 12 (6): 671-679.

As used herein, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, lentiviruses, replication defective lentiviruses, and adeno-associated viruses). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Common expression vectors of utility in recombinant DNA techniques are often in the form of plasmids. Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous viral expression vectors include retroviruses, replication defective retroviruses, adenoviruses, replication defective adenoviruses, lentiviruses, replication defective lentiviruses, and adeno-associated viruses.

It is to be inferred without explicit recitation and unless otherwise intended, that when the present disclosure relates to a polypeptide, protein, polynucleotide or antibody, a fragement an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

Applicants have provided herein the polypeptide and/or polynucleotide sequences for use in gene and protein transfer and expression techniques described below. It should be understood, although not always explicitly stated that the sequences provided herein can be used to provide the expression product as well as substantially identical sequences that produce a protein that has the same biological properties. These “biologically equivalent” or “biologically active” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polypeptide sequences are provided as examples of particular embodiments. Modifications to the sequences to amino acids with alternate amino acids that have similar charge. Additionally, an equivalent polynucleotide is one that hybridizes under stringent conditions to the reference polynucleotide or its complement or in reference to a polypeptide, a polypeptide encoded by a polynucleotide that hybridizes to the reference encoding polynucleotide under stringent conditions or its complementary strand. Alternatively, an equivalent polypeptide or protein is one that is expressed from an equivalent polynucleotide.

### Gene Editing Systems

Disclosed herein are gene editing systems involving one or more nucleotide molecules encoding a dCas9 fused to one or more transactivating proteins and one or more nucleotide molecules encoding at least one small guide RNA (sgRNA).

In some embodiment, the sgRNA has a scaffold region and a spacer region. In some embodiments, the scaffold region is an amino acid sequence that is necessary for dCas9 binding to the gRNA. In some embodiments, the spacer region hybridizes to a nucleotide sequence that is complementary to a target sequence adjacent to a 5′-end of a protospacer adjacent motif (PAM). In some embodiments, the target sequence and the PAM are located at least about 2 or about 1 kilobase (kb), at least about 1.5 kb, at least about 1 kb, at least about 0.9 kb, at least about 0.8 kb, at least about 0.7 kb, at least about 0.6 kb, at least about 0.5 kb, at least about 0.4 kb, at least about 0.3 kb, at least about 0.2 kb, at least about 0.1 kb from the transcriptional start site (TSS) of the TCF4 gene. While the target sequence and the PAM are in one aspect located can be located at least about 1 kb from the transcriptional start site, it is apparent to the skilled artisan that other ranges are within the scope of this invention, e.g., the target sequence and the PAM are located from about 2 kb, or from about 1 kb to about 0.1 kb.

In some embodiments, the sgRNAs spacer sequences are selected to bind regions in the TCF4 gene that are within at least about 1 kb or about 2 kb, at least about 1.5 kb, at least about 1 kb, at least about 0.9 kb, at least about 0.8 kb, at least about 0.7 kb, at least about 0.6 kb, at least about 0.5 kb, at least about 0.4 kb, at least about 0.3 kb, at least about 0.2 kb, at least about 0.1 kb of the TCF4 transcriptional start site.

In some embodiments, the targeted sequence is a sequence in the gene promoter. The targeted sequence or a fragment thereof hybridizes to the corresponding gRNA. In one embodiment, the targeted sequence hybridizes to the corresponding gRNA without any mismatches. In another embodiment, the targeted sequence hybridizes to the corresponding gRNA with 1, 2, 3, 4, 5, 6, 7, 8, 9, 10 or more mismatches. Based on the targeted sequence, the gRNA sequence can be determined. In one embodiment, a gRNA comprises, or consists essentially of, or yet further consists of a sequence complement to a targeted sequence, such as those as disclosed herein, or an equivalent that is capable of binding to the same targeted sequence but comprises 1, 2, 3, 4, 5, 6, 7, 8, 9, 10 or more mismatches. In another embodiment, a gRNA comprises, or consists essentially of, or yet further consists of a sequence reverse-complement to a targeted sequence, such as those as disclosed herein, or an equivalent that is capable of binding to the same targeted sequence but comprises 1, 2, 3, 4, 5, 6, 7, 8, 9, 10 or more mismatches. In yet another embodiment, a gRNA comprises, or consists essentially of, or yet further consists of a sequence reverse to a targeted sequence, such as those as disclosed herein, or an equivalent that is capable of binding to the same targeted sequence but comprises 1, 2, 3, 4, 5, 6, 7, 8, 9, 10 or more mismatches.

Transcription factors act through a DNA-binding domain that localizes a protein to a specific site within the genome and through accessory effector domains that either activate or repress transcription at or near that site.

In some embodiments, the spacer sequence of the sgRNA has the nucleic acid sequence GCATCCTAGCCTGCCCGACC (SEQ ID NO:1) or GTGCCTCCGAGGGATGCAAC (SEQ ID NO:2). In some embodiments, the system involves two sgRNAs, the first having the nucleic acid sequence GCATCCTAGCCTGCCCGACC (SEQ ID NO:1) and the second having the nucleic acid sequence GTGCCTCCGAGGGATGCAAC (SEQ ID NO:2).

In some embodiments, the spacer sequence of the sgRNA binds the transcription start site 1a and is selected from the group consisting of: TAGCATCCTGAAATGGTAGG (SEQ ID NO: 3), AGATAGCATCCTGAAATGGT (SEQ ID NO:4), AGCTCGAGCTCCTTTTCTTT (SEQ ID NO: 5), GGCAGAGCAGGCTGGTTTTT (SEQ ID NO:6), CATTTTCCTCCTACCATTTC (SEQ ID NO: 7), TTTTCTGAGACCCAAAGAAA (SEQ ID NO:8), GCTGAGATAGCATCCTGAAA (SEQ ID NO: 9), AGCCGAAGAGAAAGGGATCT (SEQ ID NO:10), AACCTAGATCCCTTTCTCTT (SEQ ID NO:11), AGAATGGGCACTGTTCGAAA (SEQ ID NO:12), TGTGTAACCCAGCATGCTGT (SEQ ID NO: 13), AGAAGGAAGCCGAAGAGAAA (SEQ ID NO: 14), ATGCTGCACAAGTGTTACCA (SEQ ID NO:15), CTCAGAAAATGTGCTTCTCT (SEQ ID NO: 16), ACTGTAGACATCAAGAGAAT (SEQ ID NO: 17), TCTCAGAAAATGTGCTTCTC (SEQ ID NO: 18), TACTGTAGACATCAAGAGAA (SEQ ID NO:19), TGTGCTTCTCTGGGCAGAGC (SEQ ID NO:20), AATATGTACATTAATTCTCT (SEQ ID NO: 21), and AGACTCTTCCAGCTTTGCCA (SEQ ID NO:22).

In some embodiments, the spacer sequence of the sgRNA binds the transcription start site 3a and is selected from the group consisting of: GCAACACGAATAAGAGCGCA (SEQ ID NO: 23), TCGCGCGTGGGGCGGCACTG (SEQ ID NO:24), GGCAACACGAATAAGAGCGC (SEQ ID NO:25), CCGAGGCCGAGGTCGTTCGC (SEQ ID NO:26), TCTCGGGTAGGCGTCGCGCG (SEQ ID NO:27), CTGGTCGACCACGCCTCCTC (SEQ ID NO: 28), CTCGGGTAGGCGTCGCGCGT (SEQ ID NO:29), TCGGGTAGGCGTCGCGCGTG (SEQ ID NO:30), GCGCTCTGTTTACCACTCTA (SEQ ID NO:31), CTGCGAACGACCTCGGCCTC (SEQ ID NO:32), TTATTCGTGTTGCCGCTTCT (SEQ ID NO: 33), ATGGGGCCTGCGAACGACCT (SEQ ID NO:34), GCGGGTGGCTGTTCTCGGGT (SEQ ID NO:35), GGTAGGCGTCGCGCGTGGGG (SEQ ID NO:36), ACTGCGGGCTTATAAAGAGA (SEQ ID NO:37), GGTAAACAGAGCGCCTAGAG (SEQ ID NO: 38), TGGTCGACCACGCCTCCTCC (SEQ ID NO:39), GTCGACCAGCACCGCCATCT (SEQ ID NO:40), GGGAGCAGGCGACCATAGAG (SEQ ID NO:41), and CGGAGGCGGGTGGCTGTTCT (SEQ ID NO:42).

In some embodiments, the spacer sequence of the sgRNA binds the transcription start site 3b and is selected from the group consisting of: CGTTTCGGTAGTTTTGCGTT (SEQ ID NO: 43), GTTTCGGTAGTTTTGCGTTG (SEQ ID NO:44), CGGCAAAGCAAGTTTATACT (SEQ ID NO:45), TCGTTTCGGTAGTTTTGCGT (SEQ ID NO:46), TTTCGGTAGTTTTGCGTTGG (SEQ ID NO:47), ACTTACAGTGCTGGCGAACA (SEQ ID NO: 48), ACCGAAACGAAAATCTCCCC (SEQ ID NO:49), AACAGGGACTTAGCGTGCCT (SEQ ID NO: 50), TAAACTTGCTTTGCCGTGTG (SEQ ID NO:51), AACTTACAGTGCTGGCGAAC (SEQ ID NO:52), TATTCTGTGACTTAAAAGGG (SEQ ID NO:53), TCCTGGGGAGATTTTCGTTT (SEQ ID NO:54), TGCAAAAAGACTTTGCCAAG (SEQ ID NO: 55), AATCAAGCCACATTTTCCTG (SEQ ID NO:56), TACTATTCTGTGACTTAAAA (SEQ ID NO: 57), TTCTGTGACTTAAAAGGGAG (SEQ ID NO:58), TAAATCAAGCCACATTTTCC (SEQ ID NO:59), GTTTTTGCACTTGAGTTCCT (SEQ ID NO:60), ATTCTGTGACTTAAAAGGGA (SEQ ID NO:61), AAATCAAGCCACATTTTCCT (SEQ ID NO: 62), GCATCCTAGCCTGCCCGACC (SEQ ID NO:1), GCCCTCGGGGTGGGTGCGAG (SEQ ID NO:63), GGCAAGTCGCGTCTCCCCGC (SEQ ID NO:64), GTGCCCGTTGCATCCCTCGG (SEQ ID NO:65), GGACTTGCTCCGGGTCGGGC (SEQ ID NO: 66), GGGGGAGACGCGACTTGCTC (SEQ ID NO:67), GCAGCAAAGAAATGGGTGGG (SEQ ID NO:68), GTGCCTCCGAGGGATGCAAC (SEQ ID NO:2), GGGGAGGGGGAAACACGCCG (SEQ ID NO:69), and GCCTCCCCGGAGAAGAGAGT (SEQ ID NO: 70).

In some embodiments, the spacer sequence of the sgRNA binds the transcription start site 3c and is selected from the group consisting of: CGGGGAGACGCGACTTGCTC (SEQ ID NO: 71), AGACGCGACTTGCTCCGGGT (SEQ ID NO:72), AGATTGTAACTGACGATCTG (SEQ ID NO:73), GACGCGACTTGCTCCGGGTC (SEQ ID NO:74), TGTCTGCGGATCTGTAGTGG (SEQ ID NO:75), GGGAAACACGCCGAGGCGCA (SEQ ID NO: 76), AACACCAACTCTCTTCTCCG (SEQ ID NO:77), ACTTGCAATTCCGTGCGCCT (SEQ ID NO: 78), GTGTGTCTGCGGATCTGTAG (SEQ ID NO:79), ACCAACTCTCTTCTCCGGGG (SEQ ID NO:80), CTGCGGATCTGTAGTGGCGG (SEQ ID NO:81), GCATCCTAGCCTGCCCGACC (SEQ ID NO:1), TCCCCTCGGGGTGGGTGCGA (SEQ ID NO: 82), GAGAGTTGGTGTTAAGAGTC (SEQ ID NO:83), CGACTTGCTCCGGGTCGGGC (SEQ ID NO:84), CCAACTCTCTTCTCCGGGGA (SEQ ID NO:85), AGCAAGTCGCGTCTCCCCGC (SEQ ID NO:86), TGCTCCGGGTCGGGCAGGCT (SEQ ID NO: 87), TGACGATCTGAGGAAAAATG (SEQ ID NO:88), and GGGGAGACGCGACTTGCTCC (SEQ ID NO:89).

In some embodiments, the spacer sequence of the sgRNA binds the transcription start site 3d and is selected from the group consisting of: TGGAGTTTTACGGCTGTACT (SEQ ID NO: 91), CATCTTACACCAAACTCATC (SEQ ID NO:92), TCCAAACCGCCTTCCAAGTG (SEQ ID NO:93), AATCCAAACCGCCTTCCAAG (SEQ ID NO:94), ATCCAAACCGCCTTCCAAGT (SEQ ID NO:95), GCCCCACTTGGAAGGCGGTT (SEQ ID NO: 96), CTCCTGACATGTCTGGGACT (SEQ ID NO:97), AAAGAGCCCCACTTGGAAGG (SEQ ID NO:98), CAGCATGAAAGAGCCCCACT (SEQ ID NO:99), ATGAAAGAGCCCCACTTGGA (SEQ ID NO:100), GACACACTTGTGGAGTTTTA (SEQ ID NO: 101), CTGGGACTTGGTTTAGGAAA (SEQ ID NO: 102), AACCAAGTCCCAGACATGTC (SEQ ID NO:103), ATTCATTCTCCTGACATGTC (SEQ ID NO:104), TTTTTAATGCCAGATGAGTT (SEQ ID NO:105), TTCATTCTCCTGACATGTCT (SEQ ID NO: 106), ACATGTCTGGGACTTGGTTT (SEQ ID NO: 107), AAAAGAAGGTCTAGAAGAGG (SEQ ID NO: 108), GGTTTAGGAAAAGGAAGCAA (SEQ ID NO: 109), and GTTTAGGAAAAGGAAGCAAA (SEQ ID NO:110).

In some embodiments, the spacer sequence of the sgRNA binds the transcription start site 5a and is selected from the group consisting of: CCTTCCTTTACGCATCAAGC (SEQ ID NO: 111), TTTGCCAGCTTGATGCGTAA (SEQ ID NO:112), ACCGTTCAACATGCCTCTCT (SEQ ID NO:113), GAGGCATGTTGAACGGTTGA (SEQ ID NO:114), TCCAAGAGAGGCATGTTGAA (SEQ ID NO: 115), GTATATATCAGTCAGTAGAA (SEQ ID NO: 116), TGTATATATCAGTCAGTAGA (SEQ ID NO:117), GCAAACTGTCGTCATTTCTT (SEQ ID NO:118), GCAATAAGCACAATAAAAGC (SEQ ID NO:119), GATTCCCAGAAGACTGAACA (SEQ ID NO:120), GCTGTCCTGAGACCTCTGTT (SEQ ID NO: 121), TATATATCAGTCAGTAGAAG (SEQ ID NO: 122), ACAAGATCTCTGCCAAACAG (SEQ ID NO: 123), AGTAGAAGGGGAAATGATAC (SEQ ID NO: 124), ACAGCCTTGTTCAGTCTTCT (SEQ ID NO:125), GACAGCCTTGTTCAGTCTTC (SEQ ID NO: 126), CAAACTGTCGTCATTTCTTT (SEQ ID NO:127), TTTACAAACATCTCCAAGAG (SEQ ID NO:128), CTCTGCCAAACAGAGGTCTC (SEQ ID NO:129), and TTTATTGTGCTTATTGCAGT (SEQ ID NO:130).

In some embodiments, the spacer sequence of the sgRNA binds the transcription start site 7a and is selected from the group consisting of: ACCAGATGCAAATTAATGGT (SEQ ID NO: 131), CCCTACCATTAATTTGCATC (SEQ ID NO:132), GGCGTGAATCTGCCAGCCAG (SEQ ID NO:1333), CCTATGATGGAAATTTAGGG (SEQ ID NO:134), AAGCACCAGATGCAAATTAA (SEQ ID NO:135), GACCCAAGTATGAGAATTTC (SEQ ID NO: 136), TCATCACATGTTTCCTATGA (SEQ ID NO: 137), GAACACAACATGCTGGGGCT (SEQ ID NO:138), TTCCTATGATGGAAATTTAG (SEQ ID NO:139), GCATGTTGTGTTCTGTCCTC (SEQ ID NO:140), GGACAGAACACAACATGCTG (SEQ ID NO: 141), GAGGACAGAACACAACATGC (SEQ ID NO: 142), TGTACATAGTAAAACATGTT (SEQ ID NO:143), GTTGTGTTCTGTCCTCTGGC (SEQ ID NO:144), AGGACAGAACACAACATGCT (SEQ ID NO: 145), ATGATGGAAATTTAGGGGGG (SEQ ID NO: 146), TTGCCAGAAATTCTCATACT (SEQ ID NO:147), TACATTTAAATAGTCTTGAA (SEQ ID NO: 148), TCCTATGATGGAAATTTAGG (SEQ ID NO: 149), and TTTCCTATGATGGAAATTTA (SEQ ID NO:150).

In some embodiments, the spacer sequence of the sgRNA binds the transcription start site 7b and is selected from the group consisting of: TGTGGCCGAATAGCATCGAA (SEQ ID NO: 151), ATGTGGCCGAATAGCATCGA (SEQ ID NO: 152), ATAGACCCTTCGATGCTATT (SEQ ID NO:153), CCAATAGACCCAGCAGGACA (SEQ ID NO:154), TATTGGTGGAATCTGAACTT (SEQ ID NO: 155), ATCATGATGGGGTTCACTCC (SEQ ID NO: 156), TCTGAACTTGGGTTCAGAGT (SEQ ID NO:157), TGTCCTGCTGGGTCTATTGG (SEQ ID NO:158), ATTCCACCAATAGACCCAGC (SEQ ID NO:159), TCATGATGGGGTTCACTCCT (SEQ ID NO:160), CAATAGACCCAGCAGGACAA (SEQ ID NO: 161), TGGGGTTCACTCCTGGGAAA (SEQ ID NO: 162), GCAGTCTACAGATAAAAATT (SEQ ID NO: 164), ATGGGGTTCACTCCTGGGAA (SEQ ID NO:165), AGCAGTCTACAGATAAAAAT (SEQ ID NO: 166), TCCATGATCCCTTGTCCTGC (SEQ ID NO: 167), AAAAATTGGGTTGTTAGAAT (SEQ ID NO: 168), GGAATTTACATTTGGGAAAC (SEQ ID NO:169), CCCAGCAGGACAAGGGATCA (SEQ ID NO:170), and TCATCATCTTGATCATGATG (SEQ ID NO:171).

In some embodiments, the spacer sequence of the sgRNA binds the transcription start site 8a and is selected from the group consisting of: GCCTCACGCTTACTCAGCAG (SEQ ID NO: 172), GCGGAGGTCATTAGAGTTTT (SEQ ID NO: 173), CGGAGGTCATTAGAGTTTTG (SEQ ID NO:174), GCAGAGGTCTCTGTTTTCGC (SEQ ID NO:175), TCCTCTGCTGAGTAAGCGTG (SEQ ID NO: 176), TGCAGAAGACCGTGTTCCTG (SEQ ID NO: 177), GGCGGAGGTCATTAGAGTTT (SEQ ID NO: 178), ACAGGAACACGGTCTTCTGC (SEQ ID NO:179), GCAGAAGACCGTGTTCCTGT (SEQ ID NO:180), CCAATTCCTATTAGTAAGAA (SEQ ID NO: 181), CTATTAGTAAGAAAGGATAC (SEQ ID NO: 182), AGACCGTGTTCCTGTGGGTC (SEQ ID NO:183), TTTCGCTGGGCCCCTTTTTC (SEQ ID NO:184), TGATGCTTTCACAAGTTAAG (SEQ ID NO:185), ATGATGCTTTCACAAGTTAA (SEQ ID NO: 186), TGCCATGACCATCTGCAGAA (SEQ ID NO: 187), GTTTCTGAGGGTCAAGGCGG (SEQ ID NO:188), CTGTATCCTTTCTTACTAAT (SEQ ID NO: 189), ACAGACAGACTCACACGGTC (SEQ ID NO:190), and TATTAGTAAGAAAGGATACA (SEQ ID NO:191).

In some embodiments, the spacer sequence of the sgRNA binds the transcription start site 10a and is selected from the group consisting of: TATCACAAAACCTCAATCCA (SEQ ID NO: 192), TATCTAAAGTCTTACTAAGT (SEQ ID NO: 193), AATAAAGCGTTTCTGTTGGA (SEQ ID NO:194), TTGTGCATAACCCCAGCAGA (SEQ ID NO:195), AAGCGTTTCTGTTGGAAGGG (SEQ ID NO: 196), CATAAGTTATTTGTGCTGTG (SEQ ID NO: 197), GTAAGTTTCTCCCTGGATTG (SEQ ID NO: 198), AGCGTTTCTGTTGGAAGGGG (SEQ ID NO: 199), TAAAGCGTTTCTGTTGGAAG (SEQ ID NO:200), CTTAAATAAAGCGTTTCTGT (SEQ ID NO:201), AAAGCGTTTCTGTTGGAAGG (SEQ ID NO: 202), ATAAAGCGTTTCTGTTGGAA (SEQ ID NO:203), ATATCACAAAACCTCAATCC (SEQ ID NO:204), CTTGAGTGCAGTGGATAGAG (SEQ ID NO:205), TTGGAAGGGGGGGAAATTGA (SEQ ID NO:206), TTGCTCTATTTTAACCTTTC (SEQ ID NO: 207), TAGAGTGGAGAGAACCTGAA (SEQ ID NO:208), AAAACTGAGAGATCAAATTA (SEQ ID NO:209), GTTGGAAGGGGGGGAAATTG (SEQ ID NO:210), and ATTTAAAGCCAAAATGGGGA (SEQ ID NO:211).

In some embodiments, the spacer sequence of the sgRNA binds the transcription start site 10b and is selected from the group consisting of: GTAAGCGAATGCGGGCTGCG (SEQ ID NO:212), GTACTGCGCATACACAATCC (SEQ ID NO:213), TACACAATCCCGGGCATGGG (SEQ ID NO:214), GGAAAGCGGTAAGCGAATGC (SEQ ID NO: 215), AGCGAATGCGGGCTGCGAGG (SEQ ID NO:216), GTATGCGCAGTACATGGTCT (SEQ ID NO:217), CCATATGGCCGGGCCAAGCG (SEQ ID NO:218), TAAGCGAATGCGGGCTGCGA (SEQ ID NO:219), TCAACTTTGCGCAGCGGAGC (SEQ ID NO: 220), TACTGCGCATACACAATCCC (SEQ ID NO:221), GATTGTGTATGCGCAGTACA (SEQ ID NO:2223), AAGCGAATGCGGGCTGCGAG (SEQ ID NO:224), CATATTCACCACGCTTGGCC (SEQ ID NO:225), CACGCTTGGCCCGGCCATAT (SEQ ID NO: 226), AGACGCTCAACTTTGCGCAG (SEQ ID NO:227), CGCATACACAATCCCGGGCA (SEQ ID NO:228), CTTTGCATATTCACCACGCT (SEQ ID NO:229), GAGACCATTCCTGGCGGGCG (SEQ ID NO:230), AGCGTCTGACAGCAGCGCCG (SEQ ID NO: 231), and GCATACACAATCCCGGGCAT (SEQ ID NO:232).

In some embodiments, the spacer sequence of the sgRNA binds the transcription start site 10c and is selected from the group consisting of: TATCACAAAACCTCAATCCA (SEQ ID NO: 233), TATCTAAAGTCTTACTAAGT (SEQ ID NO:234), AATAAAGCGTTTCTGTTGGA (SEQ ID NO:235), TTGTGCATAACCCCAGCAGA (SEQ ID NO:236), AAGCGTTTCTGTTGGAAGGG (SEQ ID NO:237), CATAAGTTATTTGTGCTGTG (SEQ ID NO: 238), GTAAGTTTCTCCCTGGATTG (SEQ ID NO:239), AGCGTTTCTGTTGGAAGGGG (SEQ ID NO:240), TAAAGCGTTTCTGTTGGAAG (SEQ ID NO:241), CTTAAATAAAGCGTTTCTGT (SEQ ID NO:242), AAAGCGTTTCTGTTGGAAGG (SEQ ID NO: 243), ATAAAGCGTTTCTGTTGGAA (SEQ ID NO:244), ATATCACAAAACCTCAATCC (SEQ ID NO:245), CTTGAGTGCAGTGGATAGAG (SEQ ID NO:246), TTGGAAGGGGGGGAAATTGA (SEQ ID NO:247), TTGCTCTATTTTAACCTTTC (SEQ ID NO: 248), TAGAGTGGAGAGAACCTGAA (SEQ ID NO:249), AAAACTGAGAGATCAAATTA (SEQ ID NO:250), GTTGGAAGGGGGGGAAATTG (SEQ ID NO:251), and ATTTAAAGCCAAAATGGGGA (SEQ ID NO:252).

In some embodiments, the spacer sequence of the sgRNA binds the transcription start site 10d and is selected from the group consisting of: CGCCTCATGCCTGGTCATTT (SEQ ID NO: 253), ACGCCTCATGCCTGGTCATT (SEQ ID NO:254), CCCCTGGATAAAGCTGCATC (SEQ ID NO:255), ACCAGGCATGAGGCGTCCCC (SEQ ID NO:256), ATTCTGTAAAAGAGGCATTC (SEQ ID NO:257), CCTAGGATGAGACCGGGAGG (SEQ ID NO: 258), TTAGTAGCTTCTCTCTACTT (SEQ ID NO:259), TGCCTGGTCATTTGGGTGGC (SEQ ID NO:260), CAAGAAGGAAGCTGCTAAAC (SEQ ID NO:261), CACCCAAATGACCAGGCATG (SEQ ID NO:262), GGGCCTAGGATGAGACCGGG (SEQ ID NO: 263), TTTAGTAGCTTCTCTCTACT (SEQ ID NO:264), AGGAGACCTCCTCTATTCCC (SEQ ID NO:265), AGACCTCCTCTATTCCCAGG (SEQ ID NO:266), TTCTGTAAAAGAGGCATTCA (SEQ ID NO:267), GCCTGGTCATTTGGGTGGCT (SEQ ID NO: 268), GCAAGAAGGAAGCTGCTAAA (SEQ ID NO:269), TCCAGGGGACGCCTCATGCC (SEQ ID NO:270), TCCAGATGCAGCTTTATCCA (SEQ ID NO:271), and CTCCAGATGCAGCTTTATCC (SEQ ID NO:272).

In some embodiments, each of the nucleotide molecules are integrated into one or more viral or plasmid vectors. In some embodiments, each of the nucleic acid molecules are integrated into the same vector. In other embodiments, the nucleotide molecules are integrated into separate vectors. In some embodiments, the viral vector is a selected from the group of a lentiviral vector, an adeno-associated viral (AAV) vector, or an adenoviral vector.

Split dCAS

In some embodiments, the disclosed epigenetic editing system involves (i) a first expression cassette comprising a first polynucleotide sequence encoding an N-terminal split protein, which comprises, from its N-terminus, a transcription activator, an N-terminal half of a catalytically inactive Cas9 (dCas9) protein (N-dCas9), and an N-terminal half of an intein (N-intein); and (ii) a second expression cassette comprising a second polynucleotide sequence encoding a C-terminal split protein, which comprises, from its N-terminus, a C-terminal half of the intein (C-intein), a C-terminal half of the dCas9 protein (C-dCas9), and an transcription activator. The N-terminal split protein and the C-terminal split protein, upon their production and by way of intein “splicing” mechanism, ultimately become rejoined to form the fusion protein having the three main components of transcription activator-dCas9.

In some embodiments, the system further includes a third expression cassette comprising a third polynucleotide sequence encoding at least one small guide RNA (sgRNA), optionally two or three sgRNAs, each of which comprises, or consists essentially of, or consisting of a scaffold region and a spacer region, wherein the spacer region hybridizes to a sequence complementary to a target sequence adjacent to 5′ end of a protospacer adjacent motif (PAM), with both the target sequence and the PAM located within 1 kilobase (kb) of a target gene (e.g., the TCF4 gene) transcription start site.

In some embodiments, the scaffold region is a sequence that is necessary for dCas9 binding to the gRNA. In some embodiments, the spacer region hybridizes to a nucleotide sequence that is complementary to a target sequence adjacent to a 5′-end of a protospacer adjacent motif (PAM). In some embodiments, the target sequence and the PAM are located at least about 2 or about 1 kilobase (kb), at least about 1.5 kb, at least about 1 kb, at least about 0.9 kb, at least about 0.8 kb, at least about 0.7 kb, at least about 0.6 kb, at least about 0.5 kb, at least about 0.4 kb, at least about 0.3 kb, at least about 0.2 kb, at least about 0.1 kb from the transcriptional start site (TSS) of a target gene, e.g., the CDKL5 gene. While the target sequence and the PAM are in one aspect located can be located at least about 1 kb from the transcriptional start site, it is apparent to the skilled artisan that other ranges are within the scope of this invention, e.g., the target sequence and the PAM are located from about 2 kb, or from about 1 kb to about 0.1 kb.

In the alternative, instead of being present in a third expression cassette, the third polynucleotide sequence encoding one or more sgRNA may be included in the first expression cassette. The transcription of the third polynucleotide sequence may be directed by the same or a separate promoter used in the transcription of the first polynucleotide sequence. Similarly, the third polynucleotide sequence encoding one or more sgRNA may be included in the second expression cassette, either instead of the first expression cassette or in addition to the first expression cassette.

In some embodiments, the first polynucleotide sequence encodes an N-terminal split protein having (from the N-terminus) the main components of a transcription activator, an N-dCas9, and an N-intein, whereas the second polynucleotide sequence encodes a C-terminal split protein having (from the N-terminus) the main components of a C-intein, a C-dCas9, and an epigenetic modifier. An exemplary dCas9 protein is a catalytically inactive Streptococcus pyogenes dCas9 (spdCas9) protein, which may be split into 1 to 713 (±1, 2, 3, 4, 5, 6, 7, 8, 9, 10, 15, 20, 25, 30, 40, 50, 60, 70, 80, 90, or 100) and 713 (±1, 2, 3, 4, 5, 6, 7, 8, 9, 10, 15, 20, 25, 30, 40, 50, 60, 70, 80, 90, or 100) to 1368 segments of DKKYSIGLAIGTNSVGWAVITDEYKVPSKKFKVLGNTDRHSIKKNLIGALLFDSGETAEATRLKRT ARRRYTRRKNRICYLQEIFSNEMAKVDDSFFHRLEESFLVEEDKKHERHPIFGNIVDEVAYHEK YPTIYHLRKKLVDSTDKADLRLIYLALAHMIKFRGHFLIEGDLNPDNSDVDKLFIQLVQTYNQLFE ENPINASGVDAKAILSARLSKSRRLENLIAQLPGEKKNGLFGNLIALSLGLTPNFKSNFDLAEDAK LQLSKDTYDDDLDNLLAQIGDQYADLFLAAKNLSDAILLSDILRVNTEITKAPLSASMIKRYDEHH QDLTLLKALVRQQLPEKYKEIFFDQSKNGYAGYIDGGASQEEFYKFIKPILEKMDGTEELLVKLN REDLLRKQRTFDNGSIPHQIHLGELHAILRRQEDFYPFLKDNREKIEKILTFRIPYYVGPLARGNS RFAWMTRKSEETITPWNFEEVVDKGASAQSFIERMTNFDKNLPNEKVLPKHSLLYEYFTVYNEL TKVKYVTEGMRKPAFLSGEQKKAIVDLLFKTNRKVTVKQLKEDYFKKIECFDSVEISGVEDRFNA SLGTYHDLLKIIKDKDFLDNEENEDILEDIVLTLTLFEDREMIEERLKTYAHLFDDKVMKQLKRRR YTGWGRLSRKLINGIRDKQSGKTILDFLKSDGFANRNFMQLIHDDSLTFKEDIQKAQVSGQGDS LHEHIANLAGSPAIKKGILQTVKVVDELVKVMGRHKPENIVIEMARENQTTQKGQKNSRERMKRI EEGIKELGSQILKEHPVENTQLQNEKLYLYYLQNGRDMYVDQELDINRLSDYDVDAIVPQSFLK DDSIDNKVLTRSDKNRGKSDNVPSEEVVKKMKNYWRQLLNAKLITQRKFDNLTKAERGGLSEL DKAGFIKRQLVETRQITKHVAQILDSRMNTKYDENDKLIREVKVITLKSKLVSDFRKDFQFYKVR EINNYHHAHDAYLNAVVGTALIKKYPKLESEFVYGDYKVYDVRKMIAKSEQEIGKATAKYFFYSN IMNFFKTEITLANGEIRKRPLIETNGETGEIVWDKGRDFATVRKVLSMPQVNIVKKTEVQTGGFS KESILPKRNSDKLIARKKDWDPKKYGGFDSPTVAYSVLVVAKVEKGKSKKLKSVKELLGITIMER SSFEKNPIDFLEAKGYKEVKKDLIIKLPKYSLFELENGRKRMLASAGELQKGNELALPSKYVNFL YLASHYEKLKGSPEDNEQKQLFVEQHKHYLDEIIEQISEFSKRVILADANLDKVLSAYNKHRDKPI REQAENIIHLFTLTNLGAPAAFKYFDTTIDRKRYTSTKEVLDATLIHQSITGLYETRIDLSQLGGD (SEQ ID NO:273) (dCas9 protein sequence) as N-dCas9 and C-dCas9, respectively.

An exemplary intein is Rhodothermus marinus (Rma) DNA helicase DnaB, which may be split into 1 to 102 (±1, 2, 3, 4, 5, 6, 7, 8, 9, 10, 12, 15, 20, 25, or 30) and 103 (±1, 2, 3, 4, 5, 6, 7, 8, 9, 10, 12, 15, 20, 25, or 30) to 154 segments of CLAGDTLITLADGRRVPIRELVSQQNFSVWALNPQTYRLERARVSRAFCTGIKPVYRLTTRLGR SIRATANHRFLTPQGWKRVDELQPGDYLALPRRIPTASMAAACPELRQLAQSDVYWDPIVSIEP DGVEEVFDLTVPGPHNFVANDIIAHN (SEQ ID NO:274) (RRmaDnaB intein sequence) as N-intein and C-intein, respectively.

Therefore, in some embodiments, the split Cas9 has the amino acid sequences:

(SpdCas9N-RmaDnaB, SEQ ID NO: 275)
DKKYSIGLAIGTNSVGWAVITDEYKVPSKKFKVLGNTDRHSIKKNLIGALLFDSGETAEATRLKRT
ARRRYTRRKNRICYLQEIFSNEMAKVDDSFFHRLEESFLVEEDKKHERHPIFGNIVDEVAYHEK
YPTIYHLRKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNFDLAEDAK
LQLSKDTYDDDLDNLLAQIGDQYADLFLAAKNLSDAILLSDILRVNTEITKAPLSASMIKRYDEHH
QDLTLLKALVRQQLPEKYKEIFFDQSKNGYAGYIDGGASQEEFYKFIKPILEKMDGTEELLVKLN
REDLLRKQRTFDNGSIPHQIHLGELHAILRRQEDFYPFLKDNREKIEKILTFRIPYYVGPLARGNS
RFAWMTRKSEETITPWNFEEVVDKGASAQSFIERMTNFDKNLPNEKVLPKHSLLYEYFTVYNEL
TKVKYVTEGMRKPAFLSGEQKKAIVDLLFKTNRKVTVKQLKEDYFKKIECFDSVEISGVEDRFNA
SLGTYHDLLKIIKDKDFLDNEENEDILEDIVLTLTLFEDREMIEERLKTYAHLFDDKVMKQLKRRR
YTGWGRLSRKLINGIRDKQSGKTILDFLKSDGFANRNFMQLIHDDSLTFKEDIQKAQVCLAGDTL
ITLADGRRVPIRELVSQQNFSVWALNPQTYRLERARVSRAFCTGIKPVYRLTTRLGRSIRATAN
HRFLTPQGWKRVDELQPGDYLALPRRIPTAS
and
(RmaDnaB-SpdCas9C, SEQ ID NO: 276)
MAAACPELRQLAQSDVYWDPIVSIEPDGVEEVFDLTVPGPHNFVANDIIAHNSGQGDSLHEHIA
NLAGSPAIKKGILQTVKVVDELVKVMGRHKPENIVIEMARENQTTQKGQKNSRERMKRIEEGIK
ELGSQILKEHPVENTQLQNEKLYLYYLQNGRDMYVDQELDINRLSDYDVDAIVPQSFLKDDSID
NKVLTRSDKNRGKSDNVPSEEVVKKMKNYWRQLLNAKLITQRKFDNLTKAERGGLSELDKAGF
IKRQLVETRQITKHVAQILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKATAKYFFYSNIMNFFK
TEITLANGEIRKRPLIETNGETGEIVWDKGRDFATVRKVLSMPQVNIVKKTEVQTGGFSKESILP
KRNSDKLIARKKDWDPKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNELALPSKYVNFLYLASH
YEKLKGSPEDNEQKQLFVEQHKHYLDEIIEQISEFSKRVILADANLDKVLSAYNKHRDKPIREQA
ENIIHLFTLTNLGAPAAFKYFDTTIDRKRYTSTKEVLDATLIHQSITGLYETRIDLSQLGGDSRAD,

### Vector Systems

Also disclosed herein are vectors containing one or more of the nucleotide molecule(s) as disclosed herein.

In some embodiments, the nucleotide molecule(s) are inserted into a viral vector. For example, the viral vector can selected from the group of retroviral vectors, adenovirus vectors, adeno-associated virus vectors, or alphavirus vectors. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106 (15): 6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5 (7): 823-827. In aspects where gene transfer is mediated by a retroviral vector, a vector construct refers to the polynucleotide comprising the retroviral genome or part thereof. Further details as to modern methods of vectors for use in gene transfer may be found in, for example, Kotterman et al. (2015) Viral Vectors for Gene Therapy: Translational and Clinical Outlook Annual Review of Biomedical Engineering 17. In some embodiments, the viral vector is a selected from the group of a lentiviral vector, an adeno-associated viral (AAV) vector, or an adenoviral vector.

In one aspect, the present disclosure provides a vector encoding a sgRNA. In some embodiments, the sgRNA comprises, or consists essentially of, or yet further consists of a scaffold region and a spacer region. In some embodiments, the sgRNA spacer region comprises the nucleic acid sequence GCATCCTAGCCTGCCCGACC (SEQ ID NO:1) or GTGCCTCCGAGGGATGCAAC (SEQ ID NO:2).

In some embodiments, the same or a separate vector contains a nucleotide molecule encoding the dCas9 fusion protein(s).

In a further aspect, the systems, nucleotides, nucleic acids or host cells as described herein are detectably labeled for research or other use. Detectable labels such as radionucleotides and fluorescent labels are commercially available and widely used.

### Host Cells

The present disclosure provides an isolated or engineered host cell comprising any one or more of the polynucleotides, gene editing systems and/or any one or more of the vectors as disclosed herein. In some embodiments, the host cell produces the gene editing system, the nucleotide molecule(s) and/or the vector(s). Additionally or alternatively, the host cell is an insect cell, a mammalian cell, or a bacterial cell. In some embodiment, the host cell is selected from a stem cell, an embryonic stem cell (that in one aspect is from an established cultured cell line), a progenitor cell, an IPSC, a neuronal progenitor cell, a neuronal stem cell or a stem or progenitor cell with the ability to differentiate into a neuron. The host cell can also be an egg, a sperm, a zygote, or a germline cell. In yet a further embodiment, the host cell is a mammalian cell. In one aspect the cell is a culture or primary cell from a non-human host or subject. In one aspect, the cell is a cell in need of genetic correction, e.g., a cell with inactive gene expression, as described herein. In a further aspect, the cell is a neuronal cell with dysfunctional gene expression. The cells are useful in cell assay systems and therapies as described herein.

In some embodiments, the nucleotide molecule is engineered to one or more of the chromosome(s) or chromosome sites of the host cell. In some embodiments, the host cell comprises homozygous polynucleotides. In another embodiment, the host cell comprises a heterozygous polynucleotide. In some aspects and/or embodiments of the disclosure herein, the nucleotide molecule is engineered to one or more of the chromosome(s) or chromosome site(s) of the mammalian cell.

In some embodiments, the host cell comprises gene editing systems comprising, or alternatively consisting essentially of, or yet further consisting of: (i) a first nucleotide molecule encoding a dCas9 fusion protein; and (ii) a second nucleotide molecule encoding at least one small guide RNA (sgRNA). In some embodiment, the second nucleotide molecule encoding at least one small guide RNA (sgRNA) that comprises a scaffold region and a spacer region. In some embodiment, the spacer region hybridizes to a nucleotide sequence that is complementary to a target sequence adjacent to a 5′-end of a protospacer adjacent motif (PAM). In some embodiments, the target sequence and the PAM are located at least 1 kilobase (kb) from the transcriptional start site (TSS) of the TCF4 gene.

Also disclosed herein is a pharmaceutical composition comprises a gene editing systems disclosed herein or an isolated or engineered host cell comprising any one or more of the polynucleotides, systems, vectors or host cells alone or in combination with each other and optionally additional therapeutic agents, and a carrier, optionally a pharmaceutically acceptable carrier or excipient. In some embodiments, the host cell produces the gene editing system, the nucleotide molecule(s) and/or the vector(s).

The vectors, gene editing systems and host cells can be used as in vitro assays systems to test new therapies or additions to the vectors, gene editing systems or host cells as described herein. Thus, in one aspect, provided herein is a method for increasing a gene expression such as TCF4 gene expression in a cell, comprising inserting into the cell the vectors and/or gene editing systems as described above. The cells can be samples isolated from subjects suspected of containing defective gene expression and/or a commercially available or laboratory generated cell line. The host cell can be a prokaryotic or a eukaryotic cell, non-limiting examples of such include an insect cell, a mammalian cell, or a bacterial cell. In some embodiment, the host cell is selected from an egg, a sperm, a zygote, or a germline cell. In yet a further embodiment, the host cell is a mammalian cell. In one aspect, the cell is a cell in need of genetic correction, e.g., a cell with dysfunctional gene expression, as described herein. In a further aspect, the cell is a neuronal cell with dysfunctional gene expression.

One of skill of the art can generate the host cell system with a cell or cells from a subject to determine if the therapy is useful for the subject. In additional or alternatively, additional therapies can be tested for combination therapy.

The insertion of the vectors and/or gene editing system can be in vitro, ex vivo or in vivo. When used in an animal, it can serve as an animal model to assay for combination therapies.

### Therapeutic and Diagnostic Methods

Disclosed herein is a method for treating or preventing Pitt-Hopkins Syndrome (PTHS) in a subject in need thereof comprising administering to the subject a pharmaceutical composition containing a gene editing system disclosed herein.

In one aspect, the present disclosure provides a method for increasing TCF4 gene expression in a cell or subject in need thereof that involves administering to the cell or subject a system of gene editing system disclosed herein.

In some embodiments, the system or pharmaceutical composition is administered to the subject by one or more of: an intravenous route, a subcutaneous route, an intramuscular route, an intradermal route, an intranasal route, an oral route, an intracranial route, an intrathecal route, an ocular route, an otic route, a rectal route, a vaginal route, an optic route, or an intraperitoneal route.

In some embodiments, the subject is a mammal. In some embodiments, the mammal is a non-human fetus, an infant, a juvenile, or an adult. In some embodiments, the system or pharmaceutical composition is administered to the subject by one or more of: an intravenous route, a subcutaneous route, an intramuscular route, an intradermal route, an intranasal route, an oral route, an intracranial route, an intrathecal route, an ocular route, an otic route, a rectal route, a vaginal route, an optic route, or an intraperitoneal route.

### Kits

In one aspect, the present invention provides a kit comprising or consisting essentially of, or yet further consisting of any one or more of the gene editing system, the vector, the host cell or the compositions and an optional instruction for use in activating a TCF4 allele in a subject in need thereof. In some embodiments, the kit is used for increasing TCF4 gene expression in a subject in need thereof. In some embodiments, the kit is used for treating or preventing Pitt-Hopkins Syndrome (PTHS) in a subject in need thereof. In some embodiments, a kit comprising the gene editing system of the present invention and optional instructions for use as described herein.

### EMBODIMENTS

Embodiment 1. A gene editing system comprising:
(i) a first nucleotide molecule encoding a catalytically inactive Cas9 (dCas9) protein fused to a miniaturized VP64-p65-Rta (miniVPR) transcriptional activator and further encoding an MS2-P65-HSF1 (MPH) transcriptional activation complex; and
(ii) a second nucleotide molecule encoding at least one small guide RNA (sgRNA) comprising a scaffold region and a spacer region, wherein the scaffold region comprises MS2 stem loops, wherein the spacer region hybridizes to a nucleotide sequence complementary to a target sequence adjacent to a 5′-end of a protospacer adjacent motif (PAM), and wherein the target sequence and the PAM are located within 1 kilobase (kb) of the transcriptional start site (TSS) of a TCF4 gene.

Embodiment 2. A gene editing system comprising:
(i) a first nucleotide molecule encoding a first half of a split catalytically inactive Cas9 (dCas9) protein fused to a mini VP64-p65-Rta (miniVPR) transcriptional activator and a first half of an intein;
(ii) a second nucleotide molecule encoding a second half of the split dCas9 fused to a second half of an intein and further encoding an MS2-P65-HSF1 (MPH) transcriptional activation complex; and
(iii) a third nucleotide molecule encoding at least one small guide RNA (sgRNA) comprising a scaffold region and a spacer region, wherein the scaffold region comprises MS2 stem loops, wherein the spacer region hybridizes to a nucleotide sequence complementary to a target sequence adjacent to a 5′-end of a protospacer adjacent motif (PAM), and wherein the target sequence and the PAM are located within 1 kilobase (kb) of the transcriptional start site (TSS) of a TCF4 gene,
wherein when the split dCas9 encoded by the first nucleotide is transpliced with the split dCas9 encoded by the third nucleotide produces a complete dCas9.

Embodiment 3. The system of embodiment 2, wherein the first nucleotide molecule encodes the N-terminal half of the split dCas9 protein fused to the miniVPR transcriptional activator and the N-terminal half of the intein, and wherein the second nucleotide molecule encodes the C-terminal half of the split dCas9 fused to the C-terminal half of the intein and encodes the MPH transcriptional activation complex.

Embodiment 4. The system of embodiment 2, wherein the first nucleotide molecule encodes the C-terminal half of the split dCas9 protein fused to the miniVPR transcriptional activator and the C-terminal half of the intein, and wherein the second nucleotide molecule encodes the N-terminal half of the split dCas9 fused to the N-terminal half of the intein and encodes the MPH transcriptional activation complex.

Embodiment 5. The system of embodiment 4, wherein the spacer region of the sgRNA is selected from the group consisting of SEQ ID NOs: 1 to 272.

Embodiment 6. The system of any one of embodiments 1 to 5, wherein each of the nucleotide molecules are integrated into one or more viral or plasmid vectors.

Embodiment 7. The system of embodiment 6, wherein the viral vector is a selected from the group of: a lentiviral vector, an adeno-associated viral (AAV) vector, or an adenoviral vector.

Embodiment 8. A vector encoding an sgRNA, wherein the sgRNA comprises a scaffold region and a spacer region, wherein the spacer region of the sgRNA is selected from the group consisting of SEQ ID NOs: 1 to 272.

Embodiment 9. The vector of any one of embodiment 9, wherein the vector further comprises a nucleotide molecule encoding a catalytically inactive Cas9 (dCas9) protein fused to at least one transcriptional activator.

Embodiment 10. The vector of embodiment 9, wherein the transcriptional activator comprises miniVPR or a biological active fragment thereof.

Embodiment 11. The vector of embodiment 8 or 10, wherein the transcriptional activator comprises MS2-P65-HSF1 (MPH) or a biological active fragment thereof.

Embodiment 12. The vector of any one of embodiments 8 to 11, wherein the vector further comprises a first nucleotide molecule encoding an N-terminal half of a catalytically inactive Cas9 (dCas9) protein fused to a first transcriptional activator and the N-terminal half an intein, and a second nucleotide molecule encoding an C-terminal half of a catalytically inactive Cas9 (dCas9) protein fused to a second transcriptional activator and the C-terminal half an intein.

Embodiment 13. The vector of embodiment 12, wherein the first and second transcriptional activator comprises miniVPR and MS2-P65-HSF1 (MPH) or MS2-P65-HSF1 (MPH) and miniVPR, respectively.

Embodiment 14. The vector of any one of embodiments 9 to 13, wherein the vector is a viral vector or a plasmid vector.

Embodiment 15. The vector of embodiment 14, wherein the viral vector is a lentiviral vector, an AAV vector, or an adenoviral vector.

Embodiment 16. A host cell comprising the system of any one of embodiments 1 to 8 or the vector of any of embodiments 9 to 15.

Embodiment 17. The host cell of embodiment 16, wherein the host cell comprises a prokaryotic or a eukaryotic cell.

Embodiment 18. The host cell of embodiment 17, wherein the host cell comprises a mammalian or a human cell.

Embodiment 19. The host cell of any one of embodiments 16 to 18, wherein the host cell is a cultured cell or a primary cell.

Embodiment 20. The host cell of embodiment 19, wherein the cell is a neuronal cell.

Embodiment 21. The host cell of any of embodiments 16 to 20, further comprising a therapeutic molecule.

Embodiment 22. A pharmaceutical composition comprising one or more of: the systems of embodiments 1 to 8, the vector of embodiments 9 to 15, or the host cell of any one of embodiments 16 to 21 and a pharmaceutically acceptable carrier or excipient.

Embodiment 23. A method for increasing TCF4 gene expression in a cell or subject comprising administering to the cell or subject the system of any one of embodiments 1 to 8 or the pharmaceutical composition of embodiment 22.

Embodiment 24. The method of embodiment 23, wherein the cell or subject is in need of increased TCF4 gene expression.

Embodiment 25. The method of embodiment 24, wherein the subject has been diagnosed with Pitt-Hopkins Syndrome (PTHS).

Embodiment 26. The method of any one of embodiments 23 to 25, wherein the pharmaceutical composition is administered to the subject by one or more of: an intravenous route, a subcutaneous route, an intramuscular route, an intradermal route, an intranasal route, an oral route, an intracranial route, an intrathecal route, an ocular route, an otic route, a rectal route, a vaginal route, an optic route, or an intraperitoneal route.

Embodiment 27. The method of any one of embodiments 23 to 26, wherein the subject is a human.

Embodiment 28. The method of embodiment 27, wherein the subject is a fetus, infant, or juvenile.

Embodiment 29. A method for treating or preventing Pitt-Hopkins Syndrome (PTHS) in a subject in need thereof comprising administering to the subject the pharmaceutical composition of embodiment 22.

Embodiment 30. The method of embodiment 29, wherein the pharmaceutical composition is administered to the subject by one or more of: an intravenous route, a subcutaneous route, an intramuscular route, an intradermal route, an intranasal route, an oral route, an intracranial route, an intrathecal route, an ocular route, an otic route, a rectal route, a vaginal route, an optic route, or an intraperitoneal route.

Embodiment 31. The method of embodiment 29 or 30, wherein the subject is a human.

Embodiment 32. The method of embodiment 31, wherein the subject is a fetus, infant, or juvenile.

Embodiment 33. A kit comprising the system of any one of embodiments 1 to 8 and optional instructions for use in using as described herein.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Evaluating the Potential of CRISPR Activation Therapy to Increase TCF4 Expression in Pitt-Hopkins Syndrome

Pitt-Hopkins Syndrome (PTHS) is characterized by severe intellectual disability and tendency for epilepsy and/or hypotonia. There is currently no treatment for PTHS. PTHS is caused by de novo mutations in Transcription Factor 4 (TCF4) which typically result in haploinsufficient expression. TCF4, which codes for a basic helix-loop-helix transcription factor, exhibits high expression in the central nervous system. TCF4 appears to be critical for neuronal development and neuronal function. Cre-induced knockout of TCF4 in adult mice altered firing patterns and dendritic structure in excitatory neurons, and induced memory deficits. However, the effects of PTHS may be ameliorated by restoring TCF4 expression. Cre-induced knockout of a STOP cassette in the Tcf4 gene in mice, to reinstate TCF4 expression in a PTHS model, improved activity levels, memory, and innate behaviors such as nest building. TCF4 expression reinstatement also normalized EEG abnormalities and expression of key TCF4-regulated genes related to neuronal maintenance. It is thus plausible that rescue of standard TCF4 expression could be beneficial. CRISPR activation (CRISPRa) is a valuable avenue of expression rescue by targeting the wild-type promoter of TCF4 for activation. CRISPRa is comprised of a guide RNA (gRNA) to target the promoter of the gene, a deactivated Cas9 (dCas9), and tethered transcriptional activators. gRNAs were evaluated for human TCF4 to design a CRISPRa system.

### Methods

23 gRNAs designed for the human genome were screened in HEK293T-LentiX cells. Of the guides, nine were designed from a dataset based on a predictive algorithm and 14 were designed from the ChopChop software. The gRNAs were cloned into a codon optimized gRNA expression plasmid. The gRNA plasmid was co-transfected with two other plasmids containing one half each of the dCas9-VPR-MPH complex, separated by an intein sequence. After analysis, a set of candidate lead gRNAs was selected and the same transfection method was performed on two independent iPSC-derived neuronal stem cell (NSCs) lines. In addition, patient (TCF4 KO) iPSCs and same-sex parental control iPSCs were differentiated into NSCs, for lead gRNA evaluation.

### Results

From the HEK293T transfection (for human guides) five gRNAs exhibited significant upregulation of TCF4 transcript expression. The lead guide was chosen as the guide with the greatest expression fold change from the transfection of the five guides in the healthy and patient NSCs.

### Conclusion

CRISPRa is able to significantly upregulate TCF4 across multiple cell lines. CRISPRa will be tested in multiple PTHS cell lines to evaluate functional rescue. As transduction can be a more efficient method of therapy delivery compared to transfection, transduction experiments with the lead guide will be performed.

### Example 2: Evaluating Therapies to Treat Haploinsufficiency in ADNP and Pitt Hopkins Syndrome

### Introduction

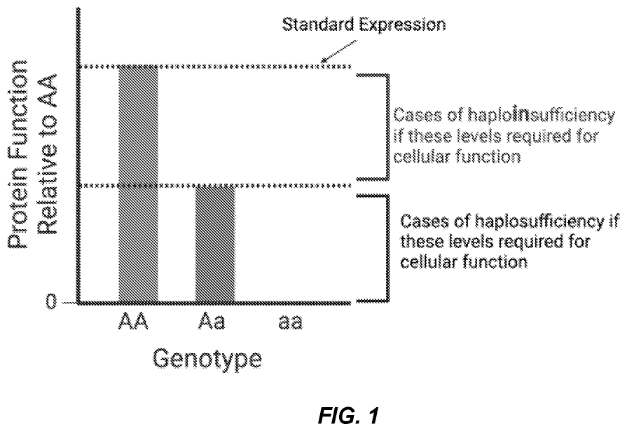

Pitt-Hopkins Syndrome (PTHS) is a disorder of intellectual disability. PTHS is caused by de novo mutations in Transcription Factor 4 (TCF4). TCF4 codes for a transcription factor that exhibits high expression in the central nervous system. It is critical for neuronal development and function. Typically the de novo mutations in TCF4 generate haploinsufficiency (FIG. 1). Haploinsufficiency occurs in an individual with one mutant allele, and the cell thus fails to meet the dosage of functional protein required for cellular function. Reinstatement of standard TCF4 expression may ameliorate symptoms of these disorders. To this end two potential avenues of expression reinstatement were identified: CRISPR activation therapy and gene replacement therapy.

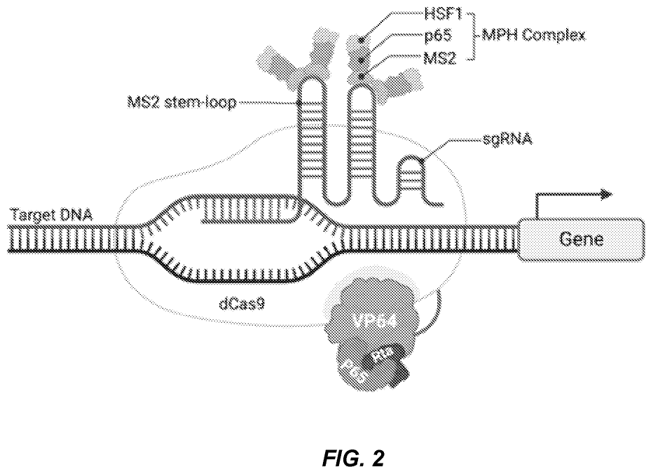

CRISPR Activation works with three components. First is a guide RNA (gRNA) to target a promoter. Second is dead Cas9 (dCas9) with deactivated nuclease activity but functional PAM recognition. The third are effector domains, i.e. transcriptional activators tethered to the dCas9. miniVPR and MPH are powerful transcriptional activators when combined in the same system (FIG. 2).

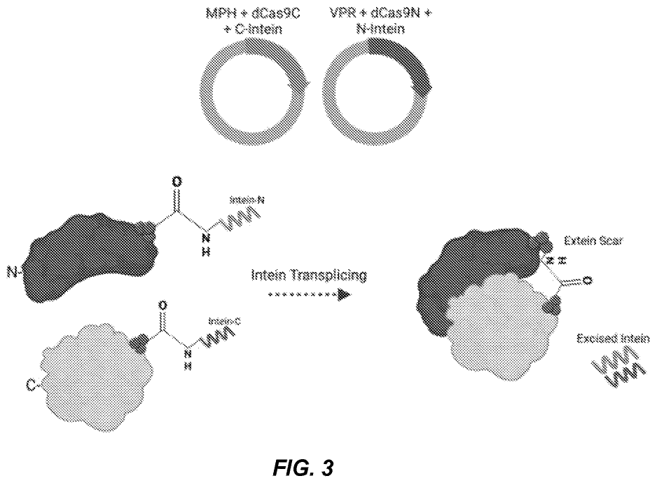

The preferred in vivo delivery of gene therapies is with AAV (Adeno-Associated Virus) capsid. However, AAVs have packaging limitations exceeded by the full sequence of CRISPRa systems. A workaround is to split the dCas9 into two sequences, in two plasmids, and attaching an intein sequence at each end (FIG. 3). After translation, the inteins auto-proteolyze to combine the C- and N-terminus of the protein, creating a whole dCas9.

### CRISPR Activation Methods

gRNA Design and Cloning

gRNAs were designed from two sources. Ten sequences were drawn from Weissman, et. al (2016) which employed a predictive algorithm to identify likely sites of gRNAs for CRISPR activation. Ten sequences were drawn from CHOP CHOP, an open-source gRNA design software. Guides labeled “C” are from CHOP CHOP, and labeled “W” are from the Weissman paper. The guides were cloned into a sgRNA guide expression plasmid. Then, NEB 5-alpha E. Coli were transformed with the plasmid, plated with carbenicillin as a selection marker, and positive colonies verified with sanger sequencing.

### Transfection

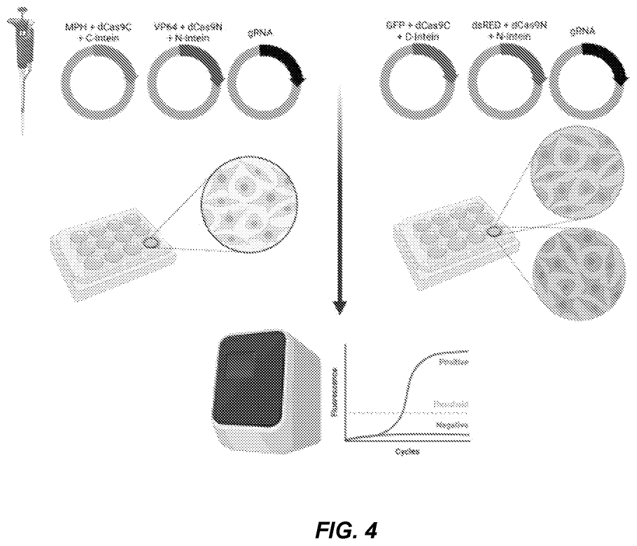

NSCs were transfected with the standard 2 μL Lipofectamine STEM protocol unless otherwise specified. The DNA transfected were three plasmids: a gRNA expressing plasmid, a dCas9 N-terminus plasmid, and a dCas9 C-terminus plasmid (FIG. 4). “miniVPR+MPH” was the treatment group, referring to a miniVPR and MPH complex coded with the N and C terminus dCas9 respectively. “No Effector” was the control group. This refers to a split dCas9 system with GFP and dsRed. 48 hours post-transfection, cells were taken down for qPCR analysis.

### Results

Human gRNA Screen

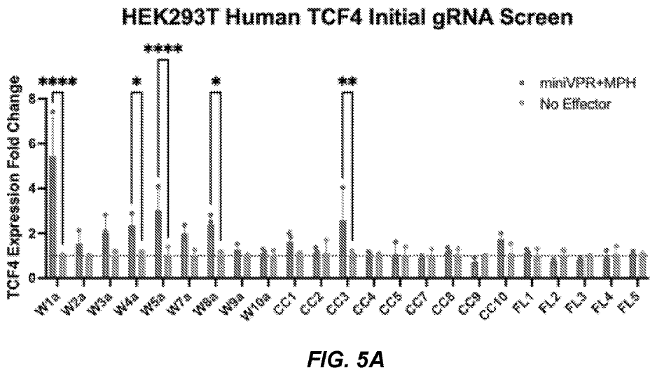

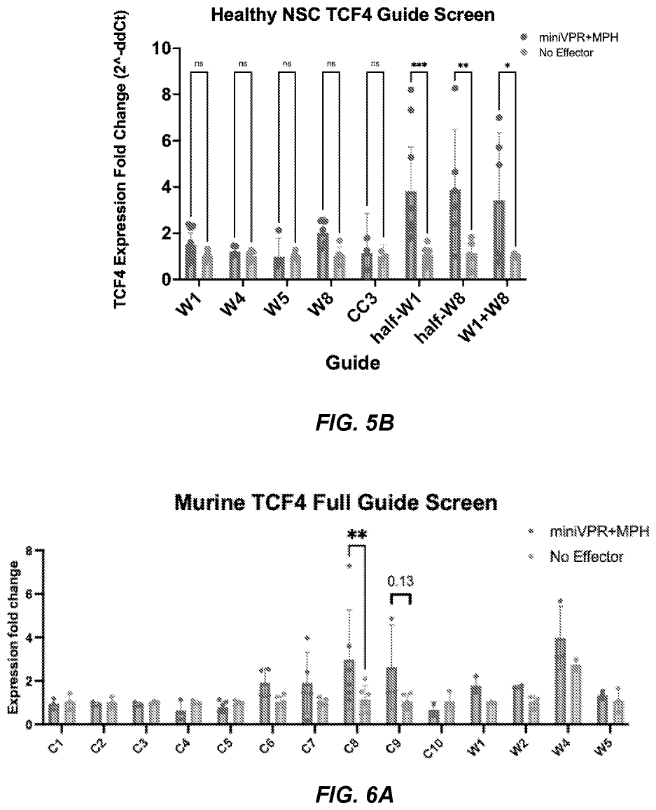

Wild-type human NSCs were transfected with gRNAs targeting the promoter of TCF4 (FIGS. 5A and 5B). qPCR was performed to identify transcript expression fold change between treatment and control (expression of TCF4 normalized to Beta Actin).

Mouse gRNA Screen

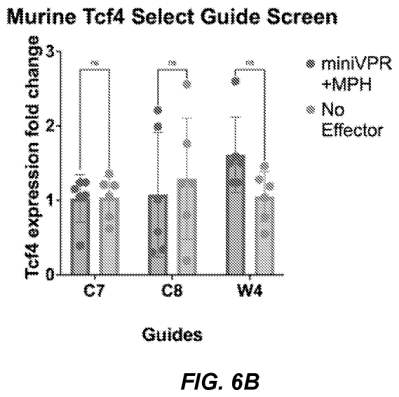

Neuro-2a cells (mouse neuroblasts) were also transfected with gRNAs targeting the promoter of TCF4 (FIGS. 6A and 6B). qPCR was performed to identify transcript expression fold change between treatment and control (expression of TCF4 normalized to Gapdh).

### Gene Replacement Therapy

Gene replacement therapy involves the insertion of a healthy, non-mutant copy of the gene of interest into the afflicted cell. One method of gene replacement involves delivery of a plasmid (coding for the healthy allele) via AAV transduction of the cell. The endogenous transcriptional machinery recognizes the promoter within the plasmid and expresses the wild type allele encoded. The expression of functional protein via this plasmid compensates for the endogenous mutant allele, thereby restoring cell function.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A gene editing system comprising:
(i) a first nucleotide molecule encoding a catalytically inactive Cas9 (dCas9) protein fused to a miniaturized VP64-p65-Rta (mini VPR) transcriptional activator and further encoding an MS2-P65-HSF1 (MPH) transcriptional activation complex; and
(ii) a second nucleotide molecule encoding at least one small guide RNA (sgRNA) comprising a scaffold region and a spacer region, wherein the scaffold region comprises MS2 stem loops, wherein the spacer region hybridizes to a nucleotide sequence complementary to a target sequence adjacent to a 5′-end of a protospacer adjacent motif (PAM), and wherein the target sequence and the PAM are located within 1 kilobase (kb) of the transcriptional start site (TSS) of a TCF4 gene.

2. A gene editing system comprising:
(i) a first nucleotide molecule encoding a first half of a split catalytically inactive Cas9 (dCas9) protein fused to a mini VP64-p65-Rta (miniVPR) transcriptional activator and a first half of an intein;
(ii) a second nucleotide molecule encoding a second half of the split dCas9 fused to a second half of an intein and further encoding an MS2-P65-HSF1 (MPH) transcriptional activation complex; and
(iii) a third nucleotide molecule encoding at least one small guide RNA (sgRNA) comprising a scaffold region and a spacer region, wherein the scaffold region comprises MS2 stem loops, wherein the spacer region hybridizes to a nucleotide sequence complementary to a target sequence adjacent to a 5′-end of a protospacer adjacent motif (PAM), and wherein the target sequence and the PAM are located within 1 kilobase (kb) of the transcriptional start site (TSS) of a TCF4 gene,
wherein when the split dCas9 encoded by the first nucleotide is transpliced with the split dCas9 encoded by the third nucleotide produces a complete dCas9.

3. The system of claim 2, wherein the first nucleotide molecule encodes the N-terminal half of the split dCas9 protein fused to the mini VPR transcriptional activator and the N-terminal half of the intein, and wherein the second nucleotide molecule encodes the C-terminal half of the split dCas9 fused to the C-terminal half of the intein and encodes the MPH transcriptional activation complex.

4. The system of claim 2, wherein the first nucleotide molecule encodes the C-terminal half of the split dCas9 protein fused to the miniVPR transcriptional activator and the C-terminal half of the intein, and wherein the second nucleotide molecule encodes the N-terminal half of the split dCas9 fused to the N-terminal half of the intein and encodes the MPH transcriptional activation complex.

5. The system of claim 4, wherein the spacer region of the sgRNA is selected from the group consisting of SEQ ID NOs: 1 to 272.

6. The system of claim 1, wherein each of the nucleotide molecules are integrated into one or more viral or plasmid vectors.

7. The system of claim 6, wherein the viral vector is a selected from the group of: a lentiviral vector, an adeno-associated viral (AAV) vector, or an adenoviral vector.

8. A vector encoding an sgRNA, wherein the sgRNA comprises a scaffold region and a spacer region, wherein the spacer region of the sgRNA is selected from the group consisting of SEQ ID NOs: 1 to 272.

9.-15. (canceled)

16. A host cell comprising the system of claim 1.

17.-21. (canceled)

22. A pharmaceutical composition comprising the systems of claim 1 and a pharmaceutically acceptable carrier or excipient.

23. A method for increasing TCF4 gene expression in a cell or subject comprising administering to the cell or subject the system of claim 1.

24. The method of claim 23, wherein the cell or subject is in need of increased TCF4 gene expression.

25. The method of claim 24, wherein the subject has been diagnosed with Pitt-Hopkins Syndrome (PTHS).

26. The method of claim 23, wherein the pharmaceutical composition is administered to the subject by one or more of: an intravenous route, a subcutaneous route, an intramuscular route, an intradermal route, an intranasal route, an oral route, an intracranial route, an intrathecal route, an ocular route, an otic route, a rectal route, a vaginal route, an optic route, or an intraperitoneal route.

27. The method of claim 23, wherein the subject is a human.

28. The method of claim 27, wherein the subject is a fetus, infant, or juvenile.

29. A method for treating or preventing Pitt-Hopkins Syndrome (PTHS) in a subject in need thereof comprising administering to the subject the pharmaceutical composition of claim 22.

30. The method of claim 29, wherein the pharmaceutical composition is administered to the subject by one or more of: an intravenous route, a subcutaneous route, an intramuscular route, an intradermal route, an intranasal route, an oral route, an intracranial route, an intrathecal route, an ocular route, an otic route, a rectal route, a vaginal route, an optic route, or an intraperitoneal route.

31. The method of claim 29, wherein the subject is a human.

32. The method of claim 31, wherein the subject is a fetus, infant, or juvenile.


SEQUENCE LISTING (276 sequences)

SEQ ID NO: 1 - RNA, 20 nt, synthetic construct
  source 1..20: mol_type = other RNA
gcatcctagcctgcccgacc

SEQ ID NO: 2 - RNA, 20 nt, synthetic construct
  source 1..20: mol_type = other RNA
gtgcctccgagggatgcaac

SEQ ID NO: 3 - RNA, 20 nt, synthetic construct
  source 1..20: mol_type = other RNA
tagcatcctgaaatggtagg

SEQ ID NO: 4 - RNA, 20 nt, synthetic construct
  source 1..20: mol_type = other RNA
agatagcatcctgaaatggt

SEQ ID NO: 5 - RNA, 20 nt, synthetic construct
  source 1..20: mol_type = other RNA
agctcgagctccttttcttt

SEQ ID NO: 6 - RNA, 20 nt, synthetic construct
  source 1..20: mol_type = other RNA
ggcagagcaggctggttttt

SEQ ID NO: 7 - RNA, 20 nt, synthetic construct
  source 1..20: mol_type = other RNA
cattttcctcctaccatttc

SEQ ID NO: 8 - RNA, 20 nt, synthetic construct
  source 1..20: mol_type = other RNA
ttttctgagacccaaagaaa

SEQ ID NO: 9 - RNA, 20 nt, synthetic construct
  source 1..20: mol_type = other RNA
gctgagatagcatcctgaaa

SEQ ID NO: 10 - RNA, 20 nt, synthetic construct
  source 1..20: mol_type = other RNA
agccgaagagaaagggatct

SEQ ID NO: 11 - RNA, 20 nt, synthetic construct
  source 1..20: mol_type = other RNA
aacctagatccctttctctt

SEQ ID NO: 12 - RNA, 20 nt, synthetic construct
  source 1..20: mol_type = other RNA
agaatgggcactgttcgaaa

SEQ ID NO: 13 - RNA, 20 nt, synthetic construct
  source 1..20: mol_type = other RNA
tgtgtaacccagcatgctgt

SEQ ID NO: 14 - RNA, 20 nt, synthetic construct
  source 1..20: mol_type = other RNA
agaaggaagccgaagagaaa

SEQ ID NO: 15 - RNA, 20 nt, synthetic construct
  source 1..20: mol_type = other RNA
atgctgcacaagtgttacca

SEQ ID NO: 16 - RNA, 20 nt, synthetic construct
  source 1..20: mol_type = other RNA
ctcagaaaatgtgcttctct

SEQ ID NO: 17 - RNA, 20 nt, synthetic construct
  source 1..20: mol_type = other RNA
actgtagacatcaagagaat

SEQ ID NO: 18 - RNA, 20 nt, synthetic construct
  source 1..20: mol_type = other RNA
tctcagaaaatgtgcttctc

SEQ ID NO: 19 - RNA, 20 nt, synthetic construct
  source 1..20: mol_type = other RNA
tactgtagacatcaagagaa

SEQ ID NO: 20 - RNA, 20 nt, synthetic construct
  source 1..20: mol_type = other RNA
tgtgcttctctgggcagagc

SEQ ID NO: 21 - RNA, 20 nt, synthetic construct
  source 1..20: mol_type = other RNA
aatatgtacattaattctct

SEQ ID NO: 22 - RNA, 20 nt, synthetic construct
  source 1..20: mol_type = other RNA
agactcttccagctttgcca

SEQ ID NO: 23 - RNA, 20 nt, synthetic construct
  source 1..20: mol_type = other RNA
gcaacacgaataagagcgca

SEQ ID NO: 24 - RNA, 20 nt, synthetic construct
  source 1..20: mol_type = other RNA
tcgcgcgtggggcggcactg

SEQ ID NO: 25 - RNA, 20 nt, synthetic construct
  source 1..20: mol_type = other RNA
ggcaacacgaataagagcgc

SEQ ID NO: 26 - RNA, 20 nt, synthetic construct
  source 1..20: mol_type = other RNA
ccgaggccgaggtcgttcgc

SEQ ID NO: 27 - RNA, 20 nt, synthetic construct
  source 1..20: mol_type = other RNA
tctcgggtaggcgtcgcgcg

SEQ ID NO: 28 - RNA, 20 nt, synthetic construct
  source 1..20: mol_type = other RNA
ctggtcgaccacgcctcctc

SEQ ID NO: 29 - RNA, 20 nt, synthetic construct
  source 1..20: mol_type = other RNA
ctcgggtaggcgtcgcgcgt

SEQ ID NO: 30 - RNA, 20 nt, synthetic construct
  source 1..20: mol_type = other RNA
tcgggtaggcgtcgcgcgtg

SEQ ID NO: 31 - RNA, 20 nt, synthetic construct
  source 1..20: mol_type = other RNA
gcgctctgtttaccactcta

SEQ ID NO: 32 - RNA, 20 nt, synthetic construct
  source 1..20: mol_type = other RNA
ctgcgaacgacctcggcctc

SEQ ID NO: 33 - RNA, 20 nt, synthetic construct
  source 1..20: mol_type = other RNA
ttattcgtgttgccgcttct

SEQ ID NO: 34 - RNA, 20 nt, synthetic construct
  source 1..20: mol_type = other RNA
atggggcctgcgaacgacct

SEQ ID NO: 35 - RNA, 20 nt, synthetic construct
  source 1..20: mol_type = other RNA
gcgggtggctgttctcgggt

SEQ ID NO: 36 - RNA, 20 nt, synthetic construct
  source 1..20: mol_type = other RNA
ggtaggcgtcgcgcgtgggg

SEQ ID NO: 37 - RNA, 20 nt, synthetic construct
  source 1..20: mol_type = other RNA
actgcgggcttataaagaga

SEQ ID NO: 38 - RNA, 20 nt, synthetic construct
  source 1..20: mol_type = other RNA
ggtaaacagagcgcctagag

SEQ ID NO: 39 - RNA, 20 nt, synthetic construct
  source 1..20: mol_type = other RNA
tggtcgaccacgcctcctcc

SEQ ID NO: 40 - RNA, 20 nt, synthetic construct
  source 1..20: mol_type = other RNA
gtcgaccagcaccgccatct

SEQ ID NO: 41 - RNA, 20 nt, synthetic construct
  source 1..20: mol_type = other RNA
gggagcaggcgaccatagag

SEQ ID NO: 42 - RNA, 20 nt, synthetic construct
  source 1..20: mol_type = other RNA
cggaggcgggtggctgttct

SEQ ID NO: 43 - RNA, 20 nt, synthetic construct
  source 1..20: mol_type = other RNA
cgtttcggtagttttgcgtt

SEQ ID NO: 44 - RNA, 20 nt, synthetic construct
  source 1..20: mol_type = other RNA
gtttcggtagttttgcgttg

SEQ ID NO: 45 - RNA, 20 nt, synthetic construct
  source 1..20: mol_type = other RNA
cggcaaagcaagtttatact

SEQ ID NO: 46 - RNA, 20 nt, synthetic construct
  source 1..20: mol_type = other RNA
tcgtttcggtagttttgcgt

SEQ ID NO: 47 - RNA, 20 nt, synthetic construct
  source 1..20: mol_type = other RNA
tttcggtagttttgcgttgg

SEQ ID NO: 48 - RNA, 20 nt, synthetic construct
  source 1..20: mol_type = other RNA
acttacagtgctggcgaaca

SEQ ID NO: 49 - RNA, 20 nt, synthetic construct
  source 1..20: mol_type = other RNA
accgaaacgaaaatctcccc

SEQ ID NO: 50 - RNA, 20 nt, synthetic construct
  source 1..20: mol_type = other RNA
aacagggacttagcgtgcct

SEQ ID NO: 51 - RNA, 20 nt, synthetic construct
  source 1..20: mol_type = other RNA
taaacttgctttgccgtgtg

SEQ ID NO: 52 - RNA, 20 nt, synthetic construct
  source 1..20: mol_type = other RNA
aacttacagtgctggcgaac

SEQ ID NO: 53 - RNA, 20 nt, synthetic construct
  source 1..20: mol_type = other RNA
tattctgtgacttaaaaggg

SEQ ID NO: 54 - RNA, 20 nt, synthetic construct
  source 1..20: mol_type = other RNA
tcctggggagattttcgttt

SEQ ID NO: 55 - RNA, 20 nt, synthetic construct
  source 1..20: mol_type = other RNA
tgcaaaaagactttgccaag

SEQ ID NO: 56 - RNA, 20 nt, synthetic construct
  source 1..20: mol_type = other RNA
aatcaagccacattttcctg

SEQ ID NO: 57 - RNA, 20 nt, synthetic construct
  source 1..20: mol_type = other RNA
tactattctgtgacttaaaa

SEQ ID NO: 58 - RNA, 20 nt, synthetic construct
  source 1..20: mol_type = other RNA
ttctgtgacttaaaagggag

SEQ ID NO: 59 - RNA, 20 nt, synthetic construct
  source 1..20: mol_type = other RNA
taaatcaagccacattttcc

SEQ ID NO: 60 - RNA, 20 nt, synthetic construct
  source 1..20: mol_type = other RNA
gtttttgcacttgagttcct

SEQ ID NO: 61 - RNA, 20 nt, synthetic construct
  source 1..20: mol_type = other RNA
attctgtgacttaaaaggga

SEQ ID NO: 62 - RNA, 20 nt, synthetic construct
  source 1..20: mol_type = other RNA
aaatcaagccacattttcct

SEQ ID NO: 63 - RNA, 20 nt, synthetic construct
  source 1..20: mol_type = other RNA
gccctcggggtgggtgcgag

SEQ ID NO: 64 - RNA, 20 nt, synthetic construct
  source 1..20: mol_type = other RNA
ggcaagtcgcgtctccccgc

SEQ ID NO: 65 - RNA, 20 nt, synthetic construct
  source 1..20: mol_type = other RNA
gtgcccgttgcatccctcgg

SEQ ID NO: 66 - RNA, 20 nt, synthetic construct
  source 1..20: mol_type = other RNA
ggacttgctccgggtcgggc

SEQ ID NO: 67 - RNA, 20 nt, synthetic construct
  source 1..20: mol_type = other RNA
gggggagacgcgacttgctc

SEQ ID NO: 68 - RNA, 20 nt, synthetic construct
  source 1..20: mol_type = other RNA
gcagcaaagaaatgggtggg

SEQ ID NO: 69 - RNA, 20 nt, synthetic construct
  source 1..20: mol_type = other RNA
ggggagggggaaacacgccg

SEQ ID NO: 70 - RNA, 20 nt, synthetic construct
  source 1..20: mol_type = other RNA
gcctccccggagaagagagt

SEQ ID NO: 71 - RNA, 20 nt, synthetic construct
  source 1..20: mol_type = other RNA
cggggagacgcgacttgctc

SEQ ID NO: 72 - RNA, 20 nt, synthetic construct
  source 1..20: mol_type = other RNA
agacgcgacttgctccgggt

SEQ ID NO: 73 - RNA, 20 nt, synthetic construct
  source 1..20: mol_type = other RNA
agattgtaactgacgatctg

SEQ ID NO: 74 - RNA, 20 nt, synthetic construct
  source 1..20: mol_type = other RNA
gacgcgacttgctccgggtc

SEQ ID NO: 75 - RNA, 20 nt, synthetic construct
  source 1..20: mol_type = other RNA
tgtctgcggatctgtagtgg

SEQ ID NO: 76 - RNA, 20 nt, synthetic construct
  source 1..20: mol_type = other RNA
gggaaacacgccgaggcgca

SEQ ID NO: 77 - RNA, 20 nt, synthetic construct
  source 1..20: mol_type = other RNA
aacaccaactctcttctccg

SEQ ID NO: 78 - RNA, 20 nt, synthetic construct
  source 1..20: mol_type = other RNA
acttgcaattccgtgcgcct

SEQ ID NO: 79 - RNA, 20 nt, synthetic construct
  source 1..20: mol_type = other RNA
gtgtgtctgcggatctgtag

SEQ ID NO: 80 - RNA, 20 nt, synthetic construct
  source 1..20: mol_type = other RNA
accaactctcttctccgggg

SEQ ID NO: 81 - RNA, 20 nt, synthetic construct
  source 1..20: mol_type = other RNA
ctgcggatctgtagtggcgg

SEQ ID NO: 82 - RNA, 20 nt, synthetic construct
  source 1..20: mol_type = other RNA
tcccctcggggtgggtgcga

SEQ ID NO: 83 - RNA, 20 nt, synthetic construct
  source 1..20: mol_type = other RNA
gagagttggtgttaagagtc

SEQ ID NO: 84 - RNA, 20 nt, synthetic construct
  source 1..20: mol_type = other RNA
cgacttgctccgggtcgggc

SEQ ID NO: 85 - RNA, 20 nt, synthetic construct
  source 1..20: mol_type = other RNA
ccaactctcttctccgggga

SEQ ID NO: 86 - RNA, 20 nt, synthetic construct
  source 1..20: mol_type = other RNA
agcaagtcgcgtctccccgc

SEQ ID NO: 87 - RNA, 20 nt, synthetic construct
  source 1..20: mol_type = other RNA
tgctccgggtcgggcaggct

SEQ ID NO: 88 - RNA, 20 nt, synthetic construct
  source 1..20: mol_type = other RNA
tgacgatctgaggaaaaatg

SEQ ID NO: 89 - RNA, 20 nt, synthetic construct
  source 1..20: mol_type = other RNA
ggggagacgcgacttgctcc

SEQ ID NO: 90 - skipped

SEQ ID NO: 91 - RNA, 20 nt, synthetic construct
  source 1..20: mol_type = other RNA
tggagttttacggctgtact

SEQ ID NO: 92 - RNA, 20 nt, synthetic construct
  source 1..20: mol_type = other RNA
catcttacaccaaactcatc

SEQ ID NO: 93 - RNA, 20 nt, synthetic construct
  source 1..20: mol_type = other RNA
tccaaaccgccttccaagtg

SEQ ID NO: 94 - RNA, 20 nt, synthetic construct
  source 1..20: mol_type = other RNA
aatccaaaccgccttccaag

SEQ ID NO: 95 - RNA, 20 nt, synthetic construct
  source 1..20: mol_type = other RNA
atccaaaccgccttccaagt

SEQ ID NO: 96 - RNA, 20 nt, synthetic construct
  source 1..20: mol_type = other RNA
gccccacttggaaggcggtt

SEQ ID NO: 97 - RNA, 20 nt, synthetic construct
  source 1..20: mol_type = other RNA
ctcctgacatgtctgggact

SEQ ID NO: 98 - RNA, 20 nt, synthetic construct
  source 1..20: mol_type = other RNA
aaagagccccacttggaagg

SEQ ID NO: 99 - RNA, 20 nt, synthetic construct
  source 1..20: mol_type = other RNA
cagcatgaaagagccccact

SEQ ID NO: 100 - RNA, 20 nt, synthetic construct
  source 1..20: mol_type = other RNA
atgaaagagccccacttgga

SEQ ID NO: 101 - RNA, 20 nt, synthetic construct
  source 1..20: mol_type = other RNA
gacacacttgtggagtttta

SEQ ID NO: 102 - RNA, 20 nt, synthetic construct
  source 1..20: mol_type = other RNA
ctgggacttggtttaggaaa

SEQ ID NO: 103 - RNA, 20 nt, synthetic construct
  source 1..20: mol_type = other RNA
aaccaagtcccagacatgtc

SEQ ID NO: 104 - RNA, 20 nt, synthetic construct
  source 1..20: mol_type = other RNA
attcattctcctgacatgtc

SEQ ID NO: 105 - RNA, 20 nt, synthetic construct
  source 1..20: mol_type = other RNA
tttttaatgccagatgagtt

SEQ ID NO: 106 - RNA, 20 nt, synthetic construct
  source 1..20: mol_type = other RNA
ttcattctcctgacatgtct

SEQ ID NO: 107 - RNA, 20 nt, synthetic construct
  source 1..20: mol_type = other RNA
acatgtctgggacttggttt

SEQ ID NO: 108 - RNA, 20 nt, synthetic construct
  source 1..20: mol_type = other RNA
aaaagaaggtctagaagagg

SEQ ID NO: 109 - RNA, 20 nt, synthetic construct
  source 1..20: mol_type = other RNA
ggtttaggaaaaggaagcaa

SEQ ID NO: 110 - RNA, 20 nt, synthetic construct
  source 1..20: mol_type = other RNA
gtttaggaaaaggaagcaaa

SEQ ID NO: 111 - RNA, 20 nt, synthetic construct
  source 1..20: mol_type = other RNA
ccttcctttacgcatcaagc

SEQ ID NO: 112 - RNA, 20 nt, synthetic construct
  source 1..20: mol_type = other RNA
tttgccagcttgatgcgtaa

SEQ ID NO: 113 - RNA, 20 nt, synthetic construct
  source 1..20: mol_type = other RNA
accgttcaacatgcctctct

SEQ ID NO: 114 - RNA, 20 nt, synthetic construct
  source 1..20: mol_type = other RNA
gaggcatgttgaacggttga

SEQ ID NO: 115 - RNA, 20 nt, synthetic construct
  source 1..20: mol_type = other RNA
tccaagagaggcatgttgaa

SEQ ID NO: 116 - RNA, 20 nt, synthetic construct
  source 1..20: mol_type = other RNA
gtatatatcagtcagtagaa

SEQ ID NO: 117 - RNA, 20 nt, synthetic construct
  source 1..20: mol_type = other RNA
tgtatatatcagtcagtaga

SEQ ID NO: 118 - RNA, 20 nt, synthetic construct
  source 1..20: mol_type = other RNA
gcaaactgtcgtcatttctt

SEQ ID NO: 119 - RNA, 20 nt, synthetic construct
  source 1..20: mol_type = other RNA
gcaataagcacaataaaagc

SEQ ID NO: 120 - RNA, 20 nt, synthetic construct
  source 1..20: mol_type = other RNA
gattcccagaagactgaaca

SEQ ID NO: 121 - RNA, 20 nt, synthetic construct
  source 1..20: mol_type = other RNA
gctgtcctgagacctctgtt

SEQ ID NO: 122 - RNA, 20 nt, synthetic construct
  source 1..20: mol_type = other RNA
tatatatcagtcagtagaag

SEQ ID NO: 123 - RNA, 20 nt, synthetic construct
  source 1..20: mol_type = other RNA
acaagatctctgccaaacag

SEQ ID NO: 124 - RNA, 20 nt, synthetic construct
  source 1..20: mol_type = other RNA
agtagaaggggaaatgatac

SEQ ID NO: 125 - RNA, 20 nt, synthetic construct
  source 1..20: mol_type = other RNA
acagccttgttcagtcttct

SEQ ID NO: 126 - RNA, 20 nt, synthetic construct
  source 1..20: mol_type = other RNA
gacagccttgttcagtcttc

SEQ ID NO: 127 - RNA, 20 nt, synthetic construct
  source 1..20: mol_type = other RNA
caaactgtcgtcatttcttt

SEQ ID NO: 128 - RNA, 20 nt, synthetic construct
  source 1..20: mol_type = other RNA
tttacaaacatctccaagag

SEQ ID NO: 129 - RNA, 20 nt, synthetic construct
  source 1..20: mol_type = other RNA
ctctgccaaacagaggtctc

SEQ ID NO: 130 - RNA, 20 nt, synthetic construct
  source 1..20: mol_type = other RNA
tttattgtgcttattgcagt

SEQ ID NO: 131 - RNA, 20 nt, synthetic construct
  source 1..20: mol_type = other RNA
accagatgcaaattaatggt

SEQ ID NO: 132 - RNA, 20 nt, synthetic construct
  source 1..20: mol_type = other RNA
ccctaccattaatttgcatc

SEQ ID NO: 133 - RNA, 20 nt, synthetic construct
  source 1..20: mol_type = other RNA
ggcgtgaatctgccagccag

SEQ ID NO: 134 - RNA, 20 nt, synthetic construct
  source 1..20: mol_type = other RNA
cctatgatggaaatttaggg

SEQ ID NO: 135 - RNA, 20 nt, synthetic construct
  source 1..20: mol_type = other RNA
aagcaccagatgcaaattaa

SEQ ID NO: 136 - RNA, 20 nt, synthetic construct
  source 1..20: mol_type = other RNA
gacccaagtatgagaatttc

SEQ ID NO: 137 - RNA, 20 nt, synthetic construct
  source 1..20: mol_type = other RNA
tcatcacatgtttcctatga

SEQ ID NO: 138 - RNA, 20 nt, synthetic construct
  source 1..20: mol_type = other RNA
gaacacaacatgctggggct

SEQ ID NO: 139 - RNA, 20 nt, synthetic construct
  source 1..20: mol_type = other RNA
ttcctatgatggaaatttag

SEQ ID NO: 140 - RNA, 20 nt, synthetic construct
  source 1..20: mol_type = other RNA
gcatgttgtgttctgtcctc

SEQ ID NO: 141 - RNA, 20 nt, synthetic construct
  source 1..20: mol_type = other RNA
ggacagaacacaacatgctg

SEQ ID NO: 142 - RNA, 20 nt, synthetic construct
  source 1..20: mol_type = other RNA
gaggacagaacacaacatgc

SEQ ID NO: 143 - RNA, 20 nt, synthetic construct
  source 1..20: mol_type = other RNA
tgtacatagtaaaacatgtt

SEQ ID NO: 144 - RNA, 20 nt, synthetic construct
  source 1..20: mol_type = other RNA
gttgtgttctgtcctctggc

SEQ ID NO: 145 - RNA, 20 nt, synthetic construct
  source 1..20: mol_type = other RNA
aggacagaacacaacatgct

SEQ ID NO: 146 - RNA, 20 nt, synthetic construct
  source 1..20: mol_type = other RNA
atgatggaaatttagggggg

SEQ ID NO: 147 - RNA, 20 nt, synthetic construct
  source 1..20: mol_type = other RNA
ttgccagaaattctcatact

SEQ ID NO: 148 - RNA, 20 nt, synthetic construct
  source 1..20: mol_type = other RNA
tacatttaaatagtcttgaa

SEQ ID NO: 149 - RNA, 20 nt, synthetic construct
  source 1..20: mol_type = other RNA
tcctatgatggaaatttagg

SEQ ID NO: 150 - RNA, 20 nt, synthetic construct
  source 1..20: mol_type = other RNA
tttcctatgatggaaattta

SEQ ID NO: 151 - RNA, 20 nt, synthetic construct
  source 1..20: mol_type = other RNA
tgtggccgaatagcatcgaa

SEQ ID NO: 152 - RNA, 20 nt, synthetic construct
  source 1..20: mol_type = other RNA
atgtggccgaatagcatcga

SEQ ID NO: 153 - RNA, 20 nt, synthetic construct
  source 1..20: mol_type = other RNA
atagacccttcgatgctatt

SEQ ID NO: 154 - RNA, 20 nt, synthetic construct
  source 1..20: mol_type = other RNA
ccaatagacccagcaggaca

SEQ ID NO: 155 - RNA, 20 nt, synthetic construct
  source 1..20: mol_type = other RNA
tattggtggaatctgaactt

SEQ ID NO: 156 - RNA, 20 nt, synthetic construct
  source 1..20: mol_type = other RNA
atcatgatggggttcactcc

SEQ ID NO: 157 - RNA, 20 nt, synthetic construct
  source 1..20: mol_type = other RNA
tctgaacttgggttcagagt

SEQ ID NO: 158 - RNA, 20 nt, synthetic construct
  source 1..20: mol_type = other RNA
tgtcctgctgggtctattgg

SEQ ID NO: 159 - RNA, 20 nt, synthetic construct
  source 1..20: mol_type = other RNA
attccaccaatagacccagc

SEQ ID NO: 160 - RNA, 20 nt, synthetic construct
  source 1..20: mol_type = other RNA
tcatgatggggttcactcct

SEQ ID NO: 161 - RNA, 20 nt, synthetic construct
  source 1..20: mol_type = other RNA
caatagacccagcaggacaa

SEQ ID NO: 162 - RNA, 20 nt, synthetic construct
  source 1..20: mol_type = other RNA
tggggttcactcctgggaaa

SEQ ID NO: 163 - skipped

SEQ ID NO: 164 - RNA, 20 nt, synthetic construct
  source 1..20: mol_type = other RNA
gcagtctacagataaaaatt

SEQ ID NO: 165 - RNA, 20 nt, synthetic construct
  source 1..20: mol_type = other RNA
atggggttcactcctgggaa

SEQ ID NO: 166 - RNA, 20 nt, synthetic construct
  source 1..20: mol_type = other RNA
agcagtctacagataaaaat

SEQ ID NO: 167 - RNA, 20 nt, synthetic construct
  source 1..20: mol_type = other RNA
tccatgatcccttgtcctgc

SEQ ID NO: 168 - RNA, 20 nt, synthetic construct
  source 1..20: mol_type = other RNA
aaaaattgggttgttagaat

SEQ ID NO: 169 - RNA, 20 nt, synthetic construct
  source 1..20: mol_type = other RNA
ggaatttacatttgggaaac

SEQ ID NO: 170 - RNA, 20 nt, synthetic construct
  source 1..20: mol_type = other RNA
cccagcaggacaagggatca

SEQ ID NO: 171 - RNA, 20 nt, synthetic construct
  source 1..20: mol_type = other RNA
tcatcatcttgatcatgatg

SEQ ID NO: 172 - RNA, 20 nt, synthetic construct
  source 1..20: mol_type = other RNA
gcctcacgcttactcagcag

SEQ ID NO: 173 - RNA, 20 nt, synthetic construct
  source 1..20: mol_type = other RNA
gcggaggtcattagagtttt

SEQ ID NO: 174 - RNA, 20 nt, synthetic construct
  source 1..20: mol_type = other RNA
cggaggtcattagagttttg

SEQ ID NO: 175 - RNA, 20 nt, synthetic construct
  source 1..20: mol_type = other RNA
gcagaggtctctgttttcgc

SEQ ID NO: 176 - RNA, 20 nt, synthetic construct
  source 1..20: mol_type = other RNA
tcctctgctgagtaagcgtg

SEQ ID NO: 177 - RNA, 20 nt, synthetic construct
  source 1..20: mol_type = other RNA
tgcagaagaccgtgttcctg

SEQ ID NO: 178 - RNA, 20 nt, synthetic construct
  source 1..20: mol_type = other RNA
ggcggaggtcattagagttt

SEQ ID NO: 179 - RNA, 20 nt, synthetic construct
  source 1..20: mol_type = other RNA
acaggaacacggtcttctgc

SEQ ID NO: 180 - RNA, 20 nt, synthetic construct
  source 1..20: mol_type = other RNA
gcagaagaccgtgttcctgt

SEQ ID NO: 181 - RNA, 20 nt, synthetic construct
  source 1..20: mol_type = other RNA
ccaattcctattagtaagaa

SEQ ID NO: 182 - RNA, 20 nt, synthetic construct
  source 1..20: mol_type = other RNA
ctattagtaagaaaggatac

SEQ ID NO: 183 - RNA, 20 nt, synthetic construct
  source 1..20: mol_type = other RNA
agaccgtgttcctgtgggtc

SEQ ID NO: 184 - RNA, 20 nt, synthetic construct
  source 1..20: mol_type = other RNA
tttcgctgggcccctttttc

SEQ ID NO: 185 - RNA, 20 nt, synthetic construct
  source 1..20: mol_type = other RNA
tgatgctttcacaagttaag

SEQ ID NO: 186 - RNA, 20 nt, synthetic construct
  source 1..20: mol_type = other RNA
atgatgctttcacaagttaa

SEQ ID NO: 187 - RNA, 20 nt, synthetic construct
  source 1..20: mol_type = other RNA
tgccatgaccatctgcagaa

SEQ ID NO: 188 - RNA, 20 nt, synthetic construct
  source 1..20: mol_type = other RNA
gtttctgagggtcaaggcgg

SEQ ID NO: 189 - RNA, 20 nt, synthetic construct
  source 1..20: mol_type = other RNA
ctgtatcctttcttactaat

SEQ ID NO: 190 - RNA, 20 nt, synthetic construct
  source 1..20: mol_type = other RNA
acagacagactcacacggtc

SEQ ID NO: 191 - RNA, 20 nt, synthetic construct
  source 1..20: mol_type = other RNA
tattagtaagaaaggataca

SEQ ID NO: 192 - RNA, 20 nt, synthetic construct
  source 1..20: mol_type = other RNA
tatcacaaaacctcaatcca

SEQ ID NO: 193 - RNA, 20 nt, synthetic construct
  source 1..20: mol_type = other RNA
tatctaaagtcttactaagt

SEQ ID NO: 194 - RNA, 20 nt, synthetic construct
  source 1..20: mol_type = other RNA
aataaagcgtttctgttgga

SEQ ID NO: 195 - RNA, 20 nt, synthetic construct
  source 1..20: mol_type = other RNA
ttgtgcataaccccagcaga

SEQ ID NO: 196 - RNA, 20 nt, synthetic construct
  source 1..20: mol_type = other RNA
aagcgtttctgttggaaggg

SEQ ID NO: 197 - RNA, 20 nt, synthetic construct
  source 1..20: mol_type = other RNA
cataagttatttgtgctgtg

SEQ ID NO: 198 - RNA, 20 nt, synthetic construct
  source 1..20: mol_type = other RNA
gtaagtttctccctggattg

SEQ ID NO: 199 - RNA, 20 nt, synthetic construct
  source 1..20: mol_type = other RNA
agcgtttctgttggaagggg

SEQ ID NO: 200 - RNA, 20 nt, synthetic construct
  source 1..20: mol_type = other RNA
taaagcgtttctgttggaag

SEQ ID NO: 201 - RNA, 20 nt, synthetic construct
  source 1..20: mol_type = other RNA
cttaaataaagcgtttctgt

SEQ ID NO: 202 - RNA, 20 nt, synthetic construct
  source 1..20: mol_type = other RNA
aaagcgtttctgttggaagg

SEQ ID NO: 203 - RNA, 20 nt, synthetic construct
  source 1..20: mol_type = other RNA
ataaagcgtttctgttggaa

SEQ ID NO: 204 - RNA, 20 nt, synthetic construct
  source 1..20: mol_type = other RNA
atatcacaaaacctcaatcc

SEQ ID NO: 205 - RNA, 20 nt, synthetic construct
  source 1..20: mol_type = other RNA
cttgagtgcagtggatagag

SEQ ID NO: 206 - RNA, 20 nt, synthetic construct
  source 1..20: mol_type = other RNA
ttggaagggggggaaattga

SEQ ID NO: 207 - RNA, 20 nt, synthetic construct
  source 1..20: mol_type = other RNA
ttgctctattttaacctttc

SEQ ID NO: 208 - RNA, 20 nt, synthetic construct
  source 1..20: mol_type = other RNA
tagagtggagagaacctgaa

SEQ ID NO: 209 - RNA, 20 nt, synthetic construct
  source 1..20: mol_type = other RNA
aaaactgagagatcaaatta

SEQ ID NO: 210 - RNA, 20 nt, synthetic construct
  source 1..20: mol_type = other RNA
gttggaagggggggaaattg

SEQ ID NO: 211 - RNA, 20 nt, synthetic construct
  source 1..20: mol_type = other RNA
atttaaagccaaaatgggga

SEQ ID NO: 212 - RNA, 20 nt, synthetic construct
  source 1..20: mol_type = other RNA
gtaagcgaatgcgggctgcg

SEQ ID NO: 213 - RNA, 20 nt, synthetic construct
  source 1..20: mol_type = other RNA
gtactgcgcatacacaatcc

SEQ ID NO: 214 - RNA, 20 nt, synthetic construct
  source 1..20: mol_type = other RNA
tacacaatcccgggcatggg

SEQ ID NO: 215 - RNA, 20 nt, synthetic construct
  source 1..20: mol_type = other RNA
ggaaagcggtaagcgaatgc

SEQ ID NO: 216 - RNA, 20 nt, synthetic construct
  source 1..20: mol_type = other RNA
agcgaatgcgggctgcgagg

SEQ ID NO: 217 - RNA, 20 nt, synthetic construct
  source 1..20: mol_type = other RNA
gtatgcgcagtacatggtct

SEQ ID NO: 218 - RNA, 20 nt, synthetic construct
  source 1..20: mol_type = other RNA
ccatatggccgggccaagcg

SEQ ID NO: 219 - RNA, 20 nt, synthetic construct
  source 1..20: mol_type = other RNA
taagcgaatgcgggctgcga

SEQ ID NO: 220 - RNA, 20 nt, synthetic construct
  source 1..20: mol_type = other RNA
tcaactttgcgcagcggagc

SEQ ID NO: 221 - RNA, 20 nt, synthetic construct
  source 1..20: mol_type = other RNA
tactgcgcatacacaatccc

SEQ ID NO: 222 - RNA, 20 nt, synthetic construct
  source 1..20: mol_type = other RNA
gattgtgtatgcgcagtaca

SEQ ID NO: 223 - skipped

SEQ ID NO: 224 - RNA, 20 nt, synthetic construct
  source 1..20: mol_type = other RNA
aagcgaatgcgggctgcgag

SEQ ID NO: 225 - RNA, 20 nt, synthetic construct
  source 1..20: mol_type = other RNA
catattcaccacgcttggcc

SEQ ID NO: 226 - RNA, 20 nt, synthetic construct
  source 1..20: mol_type = other RNA
cacgcttggcccggccatat

SEQ ID NO: 227 - RNA, 20 nt, synthetic construct
  source 1..20: mol_type = other RNA
agacgctcaactttgcgcag

SEQ ID NO: 228 - RNA, 20 nt, synthetic construct
  source 1..20: mol_type = other RNA
cgcatacacaatcccgggca

SEQ ID NO: 229 - RNA, 20 nt, synthetic construct
  source 1..20: mol_type = other RNA
ctttgcatattcaccacgct

SEQ ID NO: 230 - RNA, 20 nt, synthetic construct
  source 1..20: mol_type = other RNA
gagaccattcctggcgggcg

SEQ ID NO: 231 - RNA, 20 nt, synthetic construct
  source 1..20: mol_type = other RNA
agcgtctgacagcagcgccg

SEQ ID NO: 232 - RNA, 20 nt, synthetic construct
  source 1..20: mol_type = other RNA
gcatacacaatcccgggcat

SEQ ID NO: 233 - RNA, 20 nt, synthetic construct
  source 1..20: mol_type = other RNA
tatcacaaaacctcaatcca

SEQ ID NO: 234 - RNA, 20 nt, synthetic construct
  source 1..20: mol_type = other RNA
tatctaaagtcttactaagt

SEQ ID NO: 235 - RNA, 20 nt, synthetic construct
  source 1..20: mol_type = other RNA
aataaagcgtttctgttgga

SEQ ID NO: 236 - RNA, 20 nt, synthetic construct
  source 1..20: mol_type = other RNA
ttgtgcataaccccagcaga

SEQ ID NO: 237 - RNA, 20 nt, synthetic construct
  source 1..20: mol_type = other RNA
aagcgtttctgttggaaggg

SEQ ID NO: 238 - RNA, 20 nt, synthetic construct
  source 1..20: mol_type = other RNA
cataagttatttgtgctgtg

SEQ ID NO: 239 - RNA, 20 nt, synthetic construct
  source 1..20: mol_type = other RNA
gtaagtttctccctggattg

SEQ ID NO: 240 - RNA, 20 nt, synthetic construct
  source 1..20: mol_type = other RNA
agcgtttctgttggaagggg

SEQ ID NO: 241 - RNA, 20 nt, synthetic construct
  source 1..20: mol_type = other RNA
taaagcgtttctgttggaag

SEQ ID NO: 242 - RNA, 20 nt, synthetic construct
  source 1..20: mol_type = other RNA
cttaaataaagcgtttctgt

SEQ ID NO: 243 - RNA, 20 nt, synthetic construct
  source 1..20: mol_type = other RNA
aaagcgtttctgttggaagg

SEQ ID NO: 244 - RNA, 20 nt, synthetic construct
  source 1..20: mol_type = other RNA
ataaagcgtttctgttggaa

SEQ ID NO: 245 - RNA, 20 nt, synthetic construct
  source 1..20: mol_type = other RNA
atatcacaaaacctcaatcc

SEQ ID NO: 246 - RNA, 20 nt, synthetic construct
  source 1..20: mol_type = other RNA
cttgagtgcagtggatagag

SEQ ID NO: 247 - RNA, 20 nt, synthetic construct
  source 1..20: mol_type = other RNA
ttggaagggggggaaattga

SEQ ID NO: 248 - RNA, 20 nt, synthetic construct
  source 1..20: mol_type = other RNA
ttgctctattttaacctttc

SEQ ID NO: 249 - RNA, 20 nt, synthetic construct
  source 1..20: mol_type = other RNA
tagagtggagagaacctgaa

SEQ ID NO: 250 - RNA, 20 nt, synthetic construct
  source 1..20: mol_type = other RNA
aaaactgagagatcaaatta

SEQ ID NO: 251 - RNA, 20 nt, synthetic construct
  source 1..20: mol_type = other RNA
gttggaagggggggaaattg

SEQ ID NO: 252 - RNA, 20 nt, synthetic construct
  source 1..20: mol_type = other RNA
atttaaagccaaaatgggga

SEQ ID NO: 253 - RNA, 20 nt, synthetic construct
  source 1..20: mol_type = other RNA
cgcctcatgcctggtcattt

SEQ ID NO: 254 - RNA, 20 nt, synthetic construct
  source 1..20: mol_type = other RNA
acgcctcatgcctggtcatt

SEQ ID NO: 255 - RNA, 20 nt, synthetic construct
  source 1..20: mol_type = other RNA
cccctggataaagctgcatc

SEQ ID NO: 256 - RNA, 20 nt, synthetic construct
  source 1..20: mol_type = other RNA
accaggcatgaggcgtcccc

SEQ ID NO: 257 - RNA, 20 nt, synthetic construct
  source 1..20: mol_type = other RNA
attctgtaaaagaggcattc

SEQ ID NO: 258 - RNA, 20 nt, synthetic construct
  source 1..20: mol_type = other RNA
cctaggatgagaccgggagg

SEQ ID NO: 259 - RNA, 20 nt, synthetic construct
  source 1..20: mol_type = other RNA
ttagtagcttctctctactt

SEQ ID NO: 260 - RNA, 20 nt, synthetic construct
  source 1..20: mol_type = other RNA
tgcctggtcatttgggtggc

SEQ ID NO: 261 - RNA, 20 nt, synthetic construct
  source 1..20: mol_type = other RNA
caagaaggaagctgctaaac

SEQ ID NO: 262 - RNA, 20 nt, synthetic construct
  source 1..20: mol_type = other RNA
cacccaaatgaccaggcatg

SEQ ID NO: 263 - RNA, 20 nt, synthetic construct
  source 1..20: mol_type = other RNA
gggcctaggatgagaccggg

SEQ ID NO: 264 - RNA, 20 nt, synthetic construct
  source 1..20: mol_type = other RNA
tttagtagcttctctctact

SEQ ID NO: 265 - RNA, 20 nt, synthetic construct
  source 1..20: mol_type = other RNA
aggagacctcctctattccc

SEQ ID NO: 266 - RNA, 20 nt, synthetic construct
  source 1..20: mol_type = other RNA
agacctcctctattcccagg

SEQ ID NO: 267 - RNA, 20 nt, synthetic construct
  source 1..20: mol_type = other RNA
ttctgtaaaagaggcattca

SEQ ID NO: 268 - RNA, 20 nt, synthetic construct
  source 1..20: mol_type = other RNA
gcctggtcatttgggtggct

SEQ ID NO: 269 - RNA, 20 nt, synthetic construct
  source 1..20: mol_type = other RNA
gcaagaaggaagctgctaaa

SEQ ID NO: 270 - RNA, 20 nt, synthetic construct
  source 1..20: mol_type = other RNA
tccaggggacgcctcatgcc

SEQ ID NO: 271 - RNA, 20 nt, synthetic construct
  source 1..20: mol_type = other RNA
tccagatgcagctttatcca

SEQ ID NO: 272 - RNA, 20 nt, synthetic construct
  source 1..20: mol_type = other RNA
ctccagatgcagctttatcc

SEQ ID NO: 273 - protein, 1367 aa, unidentified
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 274 - protein, 154 aa, unidentified
CLAGDTLITLADGRRVPIRELVSQQNFSVWALNPQTYRLERARVSRAFCTGIKPVYRLTTRLGRSIRATANHRFLTPQGWKRVDELQPGDYLALPRRIPTASMAAACPELRQLAQSDVYWDPIVSIEPDGVEEVFDLTVPGPHNFVANDIIAHN

SEQ ID NO: 275 - protein, 814 aa, unidentified
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVCLAGDTLITLADGRRVPIRELVSQQNFSVWALNPQTYRLERARVSRAFCTGIKPVYRLTTRLGRSIRATANHRFLTPQGWKRVDELQPGDYLALPRRIPTAS

SEQ ID NO: 276 - protein, 711 aa, unidentified
MAAACPELRQLAQSDVYWDPIVSIEPDGVEEVFDLTVPGPHNFVANDIIAHNSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRAD